SEQUENCE LISTING (23 sequences)

SEQ ID NO: 1 - protein, 7 aa, synthetic construct
SGSYFWG

SEQ ID NO: 2 - protein, 16 aa, synthetic construct
SIYYSGITYYNPSLKS

SEQ ID NO: 3 - protein, 11 aa, synthetic construct
HDGAVAGLFDY

SEQ ID NO: 4 - protein, 11 aa, synthetic construct
GGNNIGSKSVH

SEQ ID NO: 5 - protein, 7 aa, synthetic construct
DDSDRPS

SEQ ID NO: 6 - protein, 11 aa, synthetic construct
QVWDSSSDHVV

SEQ ID NO: 7 - protein, 121 aa, synthetic construct
QLQLQESGPGLVKPSETLSLTCTVSGGSISSGSYFWGWIRQPPGKGLEWIGSIYYSGITYYNPSLKSRVTISVDTSKNQFSLKLSSVTAADTAVYYCARHDGAVAGLFDYWGQGTLVTVSS

SEQ ID NO: 8 - protein, 111 aa, synthetic construct
SYVLTQPPSVSVAPGQTARITCGGNNIGSKSVHWYQQPPGQAPVVVVYDDSDRPSGIPERFSGSNSGNTATLTISRVEAGDEAVYYCQVWDSSSDHVVFGGGTKLTVLGQP

SEQ ID NO: 9 - protein, 448 aa, synthetic construct
QLQLQESGPGLVKPSETLSLTCTVSGGSISSGSYFWGWIRQPPGKGLEWIGSIYYSGITYYNPSLKSRVTISVDTSKNQFSLKLSSVTAADTAVYYCARHDGAVAGLFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 214 aa, synthetic construct
SYVLTQPPSVSVAPGQTARITCGGNNIGSKSVHWYQQPPGQAPVVVVYDDSDRPSGIPERFSGSNSGNTATLTISRVEAGDEAVYYCQVWDSSSDHVVFGGGTKLTVLGQPKAAPSVTLFPPSSEELQANKATLVCLISDFYPGAVTVAWKGDSSPVKAGVETTTPSKQSNNKYAASSYLSLTPEQWKSHRSYSCQVTHEGSTVEKTVAPTECS

SEQ ID NO: 11 - protein, 5 aa, synthetic construct
TYAMN

SEQ ID NO: 12 - protein, 19 aa, synthetic construct
RIRSKYNNYATYYAASVKG

SEQ ID NO: 13 - protein, 14 aa, synthetic construct
HGNFGNSYVSWFAY

SEQ ID NO: 14 - protein, 14 aa, synthetic construct
RSSTGAVTTSNYAN

SEQ ID NO: 15 - protein, 7 aa, synthetic construct
GTNKRAP

SEQ ID NO: 16 - protein, 9 aa, synthetic construct
ALWYSNLWV

SEQ ID NO: 17 - protein, 125 aa, synthetic construct
EVQLVESGGGLVQPGGSLRLSCAASGFTFNTYAMNWVRQAPGKGLEWVARIRSKYNNYATYYAASVKGRFTISRDDSKNSLYLQMNSLKTEDTAVYYCARHGNFGNSYVSWFAYWGQGTLVTVSS

SEQ ID NO: 18 - protein, 112 aa, synthetic construct
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGQP

SEQ ID NO: 19 - protein, 452 aa, synthetic construct
EVQLVESGGGLVQPGGSLRLSCAASGFTFNTYAMNWVRQAPGKGLEWVARIRSKYNNYATYYAASVKGRFTISRDDSKNSLYLQMNSLKTEDTAVYYCARHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFLLYSKLTVDKSRWQEGNVFSCSVMHEALHNHYTQKSLSLSLGK

SEQ ID NO: 20 - protein, 215 aa, synthetic construct
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 21 - protein, 184 aa, synthetic construct
MLQMAGQCSQNEYFDSLLHACIPCQLRCSSNTPPLTCQRYCNASVTNSVKGTNAILWTCLGLSLIISLAVFVLMFLLRKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 22 - protein, 207 aa, synthetic construct
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 23 - protein, 104 aa, synthetic construct
DGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMD

(19) United States
(12) Patent Application Publication
(10) Pub. No.: US 2023/0279136 A1
(43) Pub. Date: Sep. 7, 2023

(54) STABLE FORMULATIONS COMPRISING A BISPECIFIC BCMA/CD3 ANTIBODY

(71) Applicant: JANSSEN BIOTECH, INC., HORSHAM, PA (US)

(72) Inventors: Salman Muzammil, Spring House, PA (US); Matthew Joseph Mistilis, Malvern, PA (US); Shyamal Choudhari, Malvern, PA (US)

(21) Appl. No.: 18/053,904

(22) Filed: Nov. 9, 2022

Related U.S. Application Data
(60) Provisional application No. 63/277,885, filed on Nov. 10, 2021.

(51) Int. Cl. C07K 16/28; A61K 9/08; A61K 47/12; A61K 47/26; A61K 47/18

(52) U.S. Cl. CPC C07K 16/2878; A61K 9/08; C07K 16/2803; A61K 47/12; A61K 47/26; A61K 47/183; C07K 2317/31

(57) ABSTRACT

Provided herein are stable aqueous pharmaceutical compositions comprising formulations of a bispecific BCMA/CD3 antibody or an antigen-binding fragment thereof and methods of preparing the same. Also provided herein are methods of treating cancer in a subject in need thereof by administering to the subject the stable aqueous pharmaceutical compositions as disclosed herein. Further provided herein are kits and articles of manufacture comprising the sable aqueous pharmaceutical compositions as disclosed herein.

### CROSS REFERENCE TO RELATED APPLICATIONS

This application claims the benefit of U.S. Provisional Application Ser. No. 63/277,885 filed 10 Nov. 2021, which is hereby incorporated by reference in its entirety.

### REFERENCE TO SEQUENCE LISTING SUBMITTED ELECTRONICALLY

This application contains a sequence listing, which is submitted electronically in XML format and is hereby incorporated by reference in its entirety. Said XML copy, created on Nov. 8, 2022 is named “258199.061001_PRD4191USNP1_SL.xml” and is 22.6 kilobytes in size.

### FIELD OF THE INVENTION

Disclosed are compositions and methods for formulating a stable pharmaceutical composition comprising bispecific BCMA/CD3 antibodies.

### BACKGROUND OF THE INVENTION

B-cell maturation antigen (BCMA), also known as CD269 and tumor necrosis factor (TNF) receptor superfamily member 17, is a receptor that plays a critical role in B lymphocytes (B cell) maturation and subsequent differentiation into plasma cells. BCMA binds 2 ligands: A proliferation-inducing ligand (APRIL; CD256) and BAFF. APRIL and BAFF are type II transmembrane proteins that are readily cleaved by Furin and secreted as soluble trimers by many cells (B cells [autocrine], monocytes, dendritic cells, T cells, osteoclasts, etc.) and can bind to the BCMA receptor. Different from other surface markers, BCMA is exclusively expressed in B-lineage cells and is selectively induced during plasma cell differentiation.

A human BCMA receptor is a 184 amino acid protein that neither has a secretory signal sequence nor any specific protease cleavage site in the N-terminal 54 amino acid extracellular domain. However, the N-terminal fragment is observed as a soluble protein in the serum as a result of gamma secretase activity that cleaves BCMA protein at the transmembrane domain. Inhibition of gamma secretase treatment results in significant increase of BCMA surface protein in human primary B-cells. High levels of soluble BCMA (sBCMA) were measured in multiple myeloma patient serum samples (data not shown) and correlated with the plasma cell counts.

BCMA mRNA and protein were universally detected in MM cell lines and in all malignant plasma cells from multiple myeloma patients by Applicants (data not shown) and others. Similarly, in multiple myeloma cell lines and patient samples, BCMA is more stably expressed compared with a key plasma cell marker (CD138) that is also expressed on normal fibroblasts and epithelial cells. BCMA expression is selective for B cell lineage and was not detected in any major tissues except for infiltrating plasma cells as determined by immunohistochemistry (IHC) methods. Taken together, the selective expression of BCMA on the B cell lineage makes it an appealing target for T-cell mediated therapy to treat plasma cell disorders like multiple myeloma.

T cell redirected killing is a desirable mode of action in many therapeutic areas. In general T cell redirecting molecules are engineered to have at least two antigen binding sites wherein one site binds a surface antigen on a target cell and the other site binds a T cell surface antigen. Amongst T cell surface antigens, the human CD3 epsilon subunit from the TCR protein complex has been the most targeted to redirect T cell killing. Various bispecific antibody formats have been shown to mediate T cell redirection in both in pre-clinical and clinical investigations.

The role of both BCMA and CD3 in cancer is well established, making these targets attractive for combination therapy. The use of anti-BCMA antibodies for the treatment of lymphomas and multiple myeloma is mentioned in WO2002066516 and WO2010104949. Antibodies against BCMA are described, e.g. in Gras M-P. et al. Int Immunol. 1997; 7:1093-1106, WO200124811, and WO200124812. Bispecific antibodies against BCMA and CD3 are described e.g. in WO2017/031104.

While anti-BCMA/CD3 antibodies have shown promising results, there remains a need in the art for pharmaceutical compositions comprising such antibodies that are stable for long periods of time at refrigerated (2-8° C.) and ambient temperatures.

### SUMMARY OF THE INVENTION

Disclosed herein are stable aqueous pharmaceutical compositions comprising specific formulations of a bispecific B-cell mature antigen (BCMA)/cluster of differentiation 3 (CD3) antibody.

In one aspect, provided herein are stable aqueous pharmaceutical compositions comprising:
(a) a concentration of about 7.5 mg/mL to about 12.5 mg/mL of a bispecific B-cell mature antigen (BCMA)/cluster of differentiation 3 (CD3) antibody or antigen-binding fragment thereof, the bispecific BCMA/CD3 antibody or antigen-binding fragment thereof comprising:
   (1) a first heavy chain (HCl) comprising a HCl variable region 1 (VH1), wherein the VH1 comprises a heavy chain complementarity determining region 1 (HCDR1), a HCDR2, and a HCDR3 having the amino acid sequences of SEQ ID NOs:1, 2, and 3, respectively;
   (2) a first light chain (LC1) comprising a LC1 variable region (VL1), wherein the VL1 comprises a light chain complementarity determining region 1 (LCDR1), a LCDR2, and a LCDR3 having the amino acid sequences of SEQ ID NOs: 4, 5, and 6, respectively;
   (3) a second heavy chain (HC2) comprising a HC2 variable region 2 (VH2), wherein the VH2 comprises a heavy chain complementarity determining region 1 (HCDR1), a HCDR2, and a HCDR3 having the amino acid sequences of SEQ ID NOs:11, 12, and 13, respectively; and
   (4) a second light chain (LC2) comprising a LC2 variable region 2 (VL2), wherein the VL2 comprises a light chain complementarity determining region 1 (LCDR1), a LCDR2, and a LCDR3 having the amino acid sequences of SEQ ID NOs:14, 15, and 16, respectively;
(b) about 10 mM to about 20 mM of acetate and/or pharmaceutically acceptable acetate salt;
(c) about 6% (w/v) to about 10% (w/v) of sucrose;
(d) about 16 μg/mL to about 24 μg/mL of ethylenediaminetetraacetic acid (EDTA);
(e) about 0.01% to about 0.07% polysorbate 20; and
(f) a pH from about 4.7 to about 5.7.

In one aspect, provided herein are stable aqueous pharmaceutical compositions comprising:
(a) a concentration of about 76.5 mg/mL to about 103.5 mg/mL of a bispecific B-cell mature antigen (BCMA)/cluster of differentiation 3 (CD3) antibody or antigen-binding fragment thereof, the bispecific BCMA/CD3 antibody or antigen-binding fragment thereof comprising:
   (1) a first heavy chain (HCl) comprising a HCl variable region 1 (VH1), wherein the VH1 comprises a heavy chain complementarity determining region 1 (HCDR1), a HCDR2, and a HCDR3 having the amino acid sequences of SEQ ID NOs:1, 2, and 3, respectively;
   (2) a first light chain (LC1) comprising a LC1 variable region (VL1), wherein the VL1 comprises a light chain complementarity determining region 1 (LCDR1), a LCDR2, and a LCDR3 having the amino acid sequences of SEQ ID NOs: 4, 5, and 6, respectively;
   (3) a second heavy chain (HC2) comprising a HC2 variable region 2 (VH2), wherein the VH2 comprises a heavy chain complementarity determining region 1 (HCDR1), a HCDR2, and a HCDR3 having the amino acid sequences of SEQ ID NOs:11, 12, and 13, respectively; and
   (4) a second light chain (LC2) comprising a LC2 variable region 2 (VL2), wherein the VL2 comprises a light chain complementarity determining region 1 (LCDR1), a LCDR2, and a LCDR3 having the amino acid sequences of SEQ ID NOs:14, 15, and 16, respectively;
(b) about 10 mM to about 20 mM of acetate and/or pharmaceutically acceptable acetate salt;
(c) about 6% (w/v) to about 10% (w/v) of sucrose;
(d) about 16 μg/mL to about 24 μg/mL of ethylenediaminetetraacetic acid (EDTA);
(e) about 0.01% to about 0.07% polysorbate 20; and
(f) a pH from about 4.7 to about 5.7.

Also provided herein are methods of treating cancer in a subject in need thereof. The methods comprise administering to the subject the stable aqueous pharmaceutical compositions, as disclosed herein.

Also provided are methods for preparing stable aqueous compositions of the bispecific B-cell mature antigen (BCMA)/cluster of differentiation 3 (CD3) antibody or antigen-binding fragment thereof. The bispecific BCMA/CD3 antibody or antigen-binding fragment thereof can, for example, comprise:
(1) a first heavy chain (HCl) comprising a HCl variable region 1 (VH1), wherein the VH1 comprises a heavy chain complementarity determining region 1 (HCDR1), a HCDR2, and a HCDR3 having the amino acid sequences of SEQ ID NOs:1, 2, and 3, respectively;
(2) a first light chain (LC1) comprising a LC1 variable region (VL1), wherein the VL1 comprises a light chain complementarity determining region 1 (LCDR1), a LCDR2, and a LCDR3 having the amino acid sequences of SEQ ID NOs: 4, 5, and 6, respectively;
(3) a second heavy chain (HC2) comprising a HC2 variable region 2 (VH2), wherein the VH2 comprises a heavy chain complementarity determining region 1 (HCDR1), a HCDR2, and a HCDR3 having the amino acid sequences of SEQ ID NOs:11, 12, and 13, respectively; and
(4) a second light chain (LC2) comprising a LC2 variable region 2 (VL2), wherein the VL2 comprises a light chain complementarity determining region 1 (LCDR1), a LCDR2, and a LCDR3 having the amino acid sequences of SEQ ID NOs:14, 15, and 16, respectively;
   The methods can, for example, comprise combining a composition comprising about 10 mg/mL or about 90 mg/mL of the bispecific BCMA/CD3 antibody, about 15 mM of acetate and/or a pharmaceutically acceptable acetate salt, about 8% (w/v) sucrose, about 20 mg/mL of EDTA, and about 0.04% polysorbate (PS) 20, wherein the stable aqueous pharmaceutical composition has a pH of about 5.2.

Also provided herein are kits comprising the stable pharmaceutical aqueous compositions, as disclosed herein, and instructions for use thereof.

Further provided are articles of manufacture comprising a container holding the stable aqueous pharmaceutical compositions, as disclosed herein.

### BRIEF DESCRIPTION OF FIGURES

The foregoing summary, as well as the following detailed description of preferred embodiments of the present application, will be better understood when read in conjunction with the appended drawings. It should be understood, however, that the application is not limited to the precise embodiments shown in the drawings.

FIG. 1 shows a graph demonstrating purity by SE-HPLC for the teclistamab acetate and histidine buffer formulations.

### DETAILED DESCRIPTION OF THE INVENTION

The disclosed compositions and methods may be understood more readily by reference to the following detailed description taken in connection with the accompanying FIGURES, which form a part of this disclosure. It is to be understood that the disclosed compositions and methods are not limited to the specific compositions and methods described and/or shown herein, and that the terminology used herein is for the purpose of describing particular embodiments by way of example only and is not intended to be limiting of the claimed compositions and methods.

Unless specifically stated otherwise, any description as to a possible mechanism or mode of action or reason for improvement is meant to be illustrative only, and the disclosed compositions and methods are not to be constrained by the correctness or incorrectness of any such suggested mechanism or mode of action or reason for improvement.

Where a range of numerical values is recited or established herein, the range includes the endpoints thereof and all the individual integers and fractions within the range, and also includes each of the narrower ranges therein formed by all the various possible combinations of those endpoints and internal integers and fractions to form subgroups of the larger group of values within the stated range to the same extent as if each of those narrower ranges was explicitly recited. Where a range of numerical values is stated herein as being greater than a stated value, the range is nevertheless finite and is bounded on its upper end by a value that is operable within the context of the invention as described herein. Where a range of numerical values is stated herein as being less than a stated value, the range is nevertheless bounded on its lower end by a non-zero value. It is not intended that the scope of the invention be limited to the specific values recited when defining a range. All ranges are inclusive and combinable.

When values are expressed as approximations, by use of the antecedent “about,” it will be understood that the particular value forms another embodiment. Reference to a particular numerical value includes at least that particular value, unless the context clearly dictates otherwise.

It is to be appreciated that certain features of the disclosed compositions and methods which are, for clarity, described herein in the context of separate embodiments, may also be provided in combination in a single embodiment. Conversely, various features of the disclosed compositions and methods that are, for brevity, described in the context of a single embodiment, may also be provided separately or in any subcombination.

As used herein, the singular forms “a,” “an,” and “the” include the plural.

Various terms relating to aspects of the description are used throughout the specification and claims. Such terms are to be given their ordinary meaning in the art unless otherwise indicated. Other specifically defined terms are to be construed in a manner consistent with the definitions provided herein.

As used herein, “about” when used in reference to numerical ranges, cutoffs, or specific values is used to indicate that the recited values may vary by up to as much as 10% from the listed value. As many of the numerical values used herein are experimentally determined, it should be understood by those skilled in the art that such determinations can, and often times will, vary among different experiments. The values used herein should not be considered unduly limiting by virtue of this inherent variation. Thus, the term “about” is used to encompass variations of ±10% or less, variations of ±5% or less, variations of ±1% or less, variations of ±0.5% or less, or variations of ±0.1% or less from the specified value.

The term “comprising” is intended to include examples encompassed by the terms “consisting essentially of” and “consisting of”; similarly, the term “consisting essentially of” is intended to include examples encompassed by the term “consisting of.” Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”.

The term “antibody,” and like terms is meant in a broad sense and includes immunoglobulin molecules or fragments thereof, including monoclonal antibodies (such as murine, human, human-adapted, humanized, and chimeric monoclonal antibodies), antibody fragments, bispecific or multispecific antibodies, dimeric, tetrameric or multimeric antibodies, and single chain antibodies.

Immunoglobulins can be assigned to five major classes, namely IgA, IgD, IgE, IgG, and IgM, depending on the heavy chain constant domain amino acid sequence. IgA and IgG are further sub-classified as the isotypes IgA1, IgA2, IgG1, IgG2, IgG3, and IgG4. Antibody light chains of any vertebrate species can be assigned to one of two clearly distinct types, namely kappa (κ) and lambda (λ), based on the amino acid sequences of their constant domains.

“Antibody fragment” refers to a portion of an immunoglobulin molecule that retains the antigen binding properties of the parental full-length antibody. Exemplary antibody fragments are heavy chain complementarity determining regions (HCDR) 1, 2, and 3, light chain complementarity determining regions (LCDR) 1, 2, and 3, a heavy chain variable region (VH), or a light chain variable region (VL). Antibody fragments include: a Fab fragment, a monovalent fragment consisting of the VL, VH, constant light (CL), and constant heavy 1 (CH1) domains; a F(ab)₂ fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; and a domain antibody (dAb) fragment (Ward et al., Nature 341:544-546, 1989), which consists of a VH domain. VH and VL domains can be engineered and linked together via a synthetic linker to form various types of single chain antibody designs where the VH/VL domains pair intramolecularly, or intermolecularly in those cases when the VH and VL domains are expressed by separate single chain antibody constructs, to form a monovalent antigen binding site, such as single chain Fv (scFv) or diabody; described for example in Int'l Pat. Pub. Nos. WO1998/44001, WO1988/01649, WO1994/13804, and WO1992/01047. These antibody fragments are obtained using techniques well known to those of skill in the art, and the fragments are screened for utility in the same manner as are full length antibodies.

An antibody variable region consists of a “framework” region interrupted by three “antigen binding sites.” The antigen binding sites are defined using various terms: (i) Complementarity Determining Regions (CDRs), three in the VH (HCDR1, HCDR2, HCDR3), and three in the VL (LCDR1, LCDR2, LCDR3) are based on sequence variability (Wu and Kabat J Exp Med 132:211-50, 1970; Kabat et al. Sequences of Proteins of Immunological Interest, 5th Ed. Public Health Service, National Institutes of Health, Bethesda, Md., 1991); and (ii) “Hypervariable regions” (“HVR” or “HV”), three in the VH (H1, H2, H3) and three in the VL (L1, L2, L3) refer to the regions of the antibody variable domains which are hypervariable in structure as defined by Chothia and Lesk (Chothia and Lesk Mol Biol 196:901-17, 1987). Other terms include “IMGT-CDRs” (Lefranc et al., Dev Comparat Immunol 27:55-77, 2003) and “Specificity Determining Residue Usage” (SDRU) (Almagro Mol Recognit 17:132-43, 2004). The International ImMunoGeneTics (IMGT) database (www_imgt_org) provides a standardized numbering and definition of antigen-binding sites. The correspondence between CDRs, HVs and IMGT delineations is described in Lefranc et al., Dev Comparat Immunol 27:55-77, 2003.

“Monoclonal antibody” refers to a preparation of antibody molecules of a single molecular composition. A monoclonal antibody composition displays a single binding specificity and affinity for a particular epitope, or in a case of a bispecific monoclonal antibody, a dual binding specificity to two distinct epitopes. Monoclonal antibody therefore refers to an antibody population with single amino acid composition in each heavy and each light chain, except for possible well-known alterations such as removal of C-terminal lysine from the antibody heavy chain. Monoclonal antibodies may have heterogeneous glycosylation within the antibody population. Monoclonal antibody may be monospecific or multispecific, or monovalent, bivalent or multivalent. A bispecific antibody is included in the term monoclonal antibody.

“Bispecific” refers to an antibody that specifically binds two distinct antigens or two distinct epitopes within the same antigen. The bispecific antibody can have cross-reactivity to other related antigens, for example to the same antigen from other species (homologs), such as human or monkey, for example Macaca cynomolgus (cynomolgus, cyno) or Pan troglodytes, or can bind an epitope that is shared between two or more distinct antigens.

“Human antibody” refers to an antibody that is optimized to have minimal immune response when administered to a human subject. Variable regions of human antibody are derived from human immunoglobulin sequences. If human antibody contains a constant region or a portion of the constant region, the constant region is also derived from human immunoglobulin sequences. Human antibody comprises heavy and light chain variable regions that are “derived from” sequences of human origin if the variable regions of the human antibody are obtained from a system that uses human germline immunoglobulin or rearranged immunoglobulin genes. Such exemplary systems are human immunoglobulin gene libraries displayed on phage, and transgenic non-human animals such as mice or rats carrying human immunoglobulin loci. “Human antibody” typically contains amino acid differences when compared to the immunoglobulins expressed in humans due to differences between the systems used to obtain the human antibody and human immunoglobulin loci, introduction of somatic mutations or intentional introduction of substitutions into the frameworks or CDRs, or both. Typically, “human antibody” is at least about 80%, 81%, 82%, 83%, 84%, 85%, 86%, 87%, 88%, 89%, 90%, 91%, 92%, 93%, 94%, 95%, 96%, 97%, 98% or 99% identical in amino acid sequence to an amino acid sequence encoded by human germline immunoglobulin or rearranged immunoglobulin genes. In some cases, “human antibody” can contain consensus framework sequences derived from human framework sequence analyses, for example as described in Knappik et al., (2000) J Mol Biol 296:57-86, or synthetic HCDR3 incorporated into human immunoglobulin gene libraries displayed on phage, for example as described in Shi et al., (2010) J Mol Biol 397:385-96, and in Int. Patent Publ. No. WO2009/085462. Antibodies in which at least one CDR is derived from a non-human species are not included in the definition of “human antibody”.

“Humanized antibody” refers to an antibody in which at least one CDR is derived from non-human species and at least one framework is derived from human immunoglobulin sequences. Humanized antibody can include substitutions in the frameworks so that the frameworks cannot be exact copies of expressed human immunoglobulin or human immunoglobulin germline gene sequences.

“Isolated” refers to a homogenous population of molecules (such as synthetic polynucleotides or a protein such as an antibody) which have been substantially separated and/or purified away from other components of the system the molecules are produced in, such as a recombinant cell, as well as a protein that has been subjected to at least one purification or isolation step. “Isolated antibody” refers to an antibody that is substantially free of other cellular material and/or chemicals and encompasses antibodies that are isolated to a higher purity, such as to 80%, 81%, 82%, 83%, 84%, 85%, 86%, 87%, 88%, 89%, 90%, 91%, 92%, 93%, 94%, 95%, 96%, 97%, 98%, 99% or 100% purity.

“BCMA/CD3 bispecific antibody” refers to a bispecific antibody that specifically binds BCMA and CD3. BCMA/CD3 bispecific antibodies are described in U.S. Pat. No. 10,072,088, which is incorporated by reference herein in its entirety.

“BCMA” refers to human B-cell maturation antigen, also known as CD269 or TNFRSF17 (UniProt Q02223). The extracellular domain of BCMA encompasses residues 1-54 of Q02223. Human BCMA comprises the amino acid sequence of SEQ ID NO: 21.

(SEQ ID NO: 21)
MLQMAGQCSQNEYFDSLLHACIPCQLRCSSNTPPLTCQRYCNASVTNSV
KGTNAILWTCLGLSLIISLAVFVLMFLLRKINSEPLKDEFKNTGSGLLG
MANIDLEKSRTGDEIILPRGLEYTVEECTCEDCIKSKPKVDSDHCFPLP
AMEEGATILVTTKTNDYCKSLPAALSATEIEKSISAR

“CD3” refers to a human antigen which is expressed on T cells as part of the multimolecular T cell receptor (TCR) complex and which consists of a homodimer or heterodimer formed from the association of two or four receptor chains: CD3 epsilon, CD3 delta, CD3 zeta and CD3 gamma. Human CD3 epsilon comprises the amino acid sequence of SEQ ID NO: 22. SEQ ID NO: 23 shows the extracellular domain of CD3 epsilon.

(SEQ ID NO: 22)
MQSGTHWRVLGLCLLSVGVWGQDGNEEMGGITQTPYKVSISGTTVILTC
PQYPGSEILWQHNDKNIGGDEDDKNIGSDEDHLSLKEFSELEQSGYYVC
YPRGSKPEDANFYLYLRARVCENCMEMDVMSVATIVIVDICITGGLLLL
VYYWSKNRKAKAKPVTRGAGAGGRQRGQNKERPPPVPNPDYEPIRKGQR
DLYSGLNQRRI
(SEQ ID NO: 23)
DGNEEMGGITQTPYKVSISGTTVILTCPQYPGSEILWQHNDKNIGGDED
DKNIGSDEDHLSLKEFSELEQSGYYVCYPRGSKPEDANFYLYLRARVCE
NCMEMD

“Epitope” refers to a portion of an antigen to which an antibody specifically binds. Epitopes usually consist of chemically active (such as polar, non-polar, or hydrophobic) surface groupings of moieties such as amino acids or polysaccharide side chains and can have specific three-dimensional structural characteristics, as well as specific charge characteristics. An epitope can be composed of contiguous and/or discontiguous amino acids that form a conformational spatial unit. For a discontiguous epitope, amino acids from differing portions of the linear sequence of the antigen come in close proximity in 3-dimensional space through the folding of the protein molecule.

“Variant” refers to a polypeptide or a polynucleotide that differs from a reference polypeptide or a reference polynucleotide by one or more modifications for example, substitutions, insertions, or deletions.

“In combination with” means that two or more therapeutics can be administered to a subject together in a mixture, concurrently as single agents, or sequentially as single agents in any order.

“Treat,” “treatment,” and like terms refer to both therapeutic treatment and prophylactic or preventative measures, and includes reducing the severity and/or frequency of symptoms, eliminating symptoms and/or the underlying cause of the symptoms, reducing the frequency or likelihood of symptoms and/or their underlying cause, improving or remediating damage caused, directly or indirectly, by the malignancy. Treatment also includes prolonging survival as compared to the expected survival of a subject not receiving treatment. Subjects to be treated include those that have the condition or disorder as well as those prone to have the condition or disorder or those in which the condition or disorder is to be prevented.

“Therapeutically effective amount” refers to an amount of the disclosed composition, which is therapeutically effective at dosages and for periods of time necessary, to achieve a desired treatment. A therapeutically effective amount may vary according to factors such as the disease state, age, sex, and weight of the subject, and the ability of the combination therapy to elicit a desired response in the subject. Exemplary indicators of a therapeutically effect amount include, for example, improved well-being of the patient, reduction of a tumor burden, arrested or slowed growth of a tumor, and/or absence of metastasis of cancer cells to other locations in the body.

“Pharmaceutical composition” refers to composition that comprises an active ingredient and a pharmaceutically acceptable carrier.

“Pharmaceutically acceptable carrier” or “excipient” refers to an ingredient in a pharmaceutical composition, other than the active ingredient, which is nontoxic to a subject.

The term “cancer” as used herein is defined as disease characterized by the rapid and uncontrolled growth of aberrant cells. Cancer cells can spread locally or through the bloodstream and lymphatic system to other parts of the body. In certain embodiments, the cancer is a hematological malignancy or a solid tumor. In some embodiments, the hematological malignancy is a multiple myeloma, a smoldering multiple myeloma, a monoclonal gammopathy of undetermined significance (MGUS), an acute lymphoblastic leukemia (ALL), a diffuse large B-cell lymphoma (DLBCL), a Burkitt's lymphoma (BL), a follicular lymphoma (FL), a mantle-cell lymphoma (MCL), Waldenstrom's macroglobulinema, a plasma cell leukemia, a light chain amyloidosis (AL), a precursor B-cell lymphoblastic leukemia, a precursor B-cell lymphoblastic leukemia, an acute myeloid leukemia (AML), a myelodysplastic syndrome (MDS), a chronic lymphocytic leukemia (CLL), a B cell malignancy, a chronic myeloid leukemia (CML), a hairy cell leukemia (HCL), a blastic plasmacytoid dendritic cell neoplasm, Hodgkin's lymphoma, non-Hodgkin's lymphoma, a marginal zone B-cell lymphoma (MZL), a mucosa-associated lymphatic tissue lymphoma (MALT), plasma cell leukemia, anaplastic large-cell lymphoma (ALCL), leukemia or lymphoma.

“Tumor cell” or a “cancer cell” refers to a cancerous, pre-cancerous or transformed cell, either in vivo, ex vivo, or in tissue culture, that has spontaneous or induced phenotypic changes. These changes do not necessarily involve the uptake of new genetic material. Although transformation can arise from infection with a transforming virus and incorporation of new genomic nucleic acid, uptake of exogenous nucleic acid or it can also arise spontaneously or following exposure to a carcinogen, thereby mutating an endogenous gene. Transformation/cancer is exemplified by morphological changes, immortalization of cells, aberrant growth control, foci formation, proliferation, malignancy, modulation of tumor specific marker levels, invasiveness, tumor growth in suitable animal hosts such as nude mice, and the like, in vitro, in vivo, and ex vivo.

“T cell redirecting therapeutic” refers to a molecule containing two or more binding regions, wherein one of the binding regions specifically binds a cell surface antigen on a target cell or tissue and wherein a second binding region of the molecule specifically binds a T cell antigen. Examples of cell surface antigen include a tumor associated antigen, such as BCMA. Examples of T cell antigen include, e.g., CD3. This dual/multi-target binding ability recruits T cells to the target cell or tissue leading to the eradication of the target cell or tissue.

“Subject” includes any human or nonhuman animal. “Nonhuman animal” includes all vertebrates, e.g., mammals and non-mammals, such as nonhuman primates, sheep, dogs, cats, horses, cows, chickens, amphibians, reptiles, etc. The terms “subject” and “patient” can be used interchangeably herein.

### DESCRIPTION

The numbering of amino acid residues in the antibody constant region throughout the specification is according to the EU index as described in Kabat et al., Sequences of Proteins of Immunological Interest, 5th Ed. Public Health Service, National Institutes of Health, Bethesda, Md. (1991), unless otherwise explicitly stated. Antibody constant chain numbering can be found for example at ImMunoGeneTics website, at IMGT Web resources at IMGT Scientific charts.

Conventional one and three-letter amino acid codes are used herein as shown in Table 1.

TABLE 1

| Amino acid | Three-letter code | One-letter code |
|---|---|---|
| Alanine | Ala | A |
| Arginine | Arg | R |
| Asparagine | Asn | N |
| Aspartate | Asp | D |
| Cysteine | Cys | C |
| Glutamate | Gln | E |
| Glutamine | Glu | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

BCMAxCD3 Bispecific Antibodies and Uses Thereof

The invention is based, at least in part, on the finding that the therapeutic agent teclistamab, a bispecific BCMA/CD3 antibody, can be used to treat multiple myeloma in subjects that are relapsed or refractory to treatment with a prior anti-cancer therapeutic.

B-cell maturation antigen (BCMA) is a cell membrane bound tumor necrosis factor receptor family member involved in differentiation of B-cells to plasma cells. Expression of BCMA is restricted to the B-cell lineage where it is predominantly expressed in the interfollicular region of germinal centers and on differentiated plasma cells and plasmablasts. BCMA is virtually absent on naïve and memory B cells (Tai and Anderson, Immunotherapy 7: 1187-99, 2015).

Thus, disclosed herein are stable, aqueous pharmaceutical compositions comprising a bispecific BCMA/CD3 antibody.

In one aspect, provided herein are stable aqueous pharmaceutical compositions comprising:
(a) a concentration of about 7.5 mg/mL to about 12.5 mg/mL of a bispecific B-cell mature antigen (BCMA)/cluster of differentiation 3 (CD3) antibody or antigen-binding fragment thereof, the bispecific BCMA/CD3 antibody or antigen-binding fragment thereof comprising:
   (1) a first heavy chain (HCl) comprising a HCl variable region 1 (VH1), wherein the VH1 comprises a heavy chain complementarity determining region 1 (HCDR1), a HCDR2, and a HCDR3 having the amino acid sequences of SEQ ID NOs:1, 2, and 3, respectively;
   (2) a first light chain (LC1) comprising a LC1 variable region (VL1), wherein the VL1 comprises a light chain complementarity determining region 1 (LCDR1), a LCDR2, and a LCDR3 having the amino acid sequences of SEQ ID NOs: 4, 5, and 6, respectively;
   (3) a second heavy chain (HC2) comprising a HC2 variable region 2 (VH2), wherein the VH2 comprises a heavy chain complementarity determining region 1 (HCDR1), a HCDR2, and a HCDR3 having the amino acid sequences of SEQ ID NOs:11, 12, and 13, respectively; and
   (4) a second light chain (LC2) comprising a LC2 variable region 2 (VL2), wherein the VL2 comprises a light chain complementarity determining region 1 (LCDR1), a LCDR2, and a LCDR3 having the amino acid sequences of SEQ ID NOs:14, 15, and 16, respectively;
(b) about 10 mM to about 20 mM of acetate and/or pharmaceutically acceptable acetate salt;
(c) about 6% (w/v) to about 10% (w/v) of sucrose;
(d) about 16 μg/mL to about 24 μg/mL of ethylenediaminetetraacetic acid (EDTA);
(e) about 0.01% to about 0.07% polysorbate 20; and
(f) a pH from about 4.7 to about 5.7.

In another general aspect, provided herein are stable aqueous pharmaceutical compositions comprising:
(a) a concentration of about 76.5 mg/mL to about 103.5 mg/mL of a bispecific B-cell mature antigen (BCMA)/cluster of differentiation 3 (CD3) antibody or antigen-binding fragment thereof, the bispecific BCMA/CD3 antibody or antigen-binding fragment thereof comprising:
   (1) a first heavy chain (HCl) comprising a HCl variable region 1 (VH1), wherein the VH1 comprises a heavy chain complementarity determining region 1 (HCDR1), a HCDR2, and a HCDR3 having the amino acid sequences of SEQ ID NOs:1, 2, and 3, respectively;
   (2) a first light chain (LC1) comprising a LC1 variable region (VL1), wherein the VL1 comprises a light chain complementarity determining region 1 (LCDR1), a LCDR2, and a LCDR3 having the amino acid sequences of SEQ ID NOs: 4, 5, and 6, respectively;
   (3) a second heavy chain (HC2) comprising a HC2 variable region 2 (VH2), wherein the VH2 comprises a heavy chain complementarity determining region 1 (HCDR1), a HCDR2, and a HCDR3 having the amino acid sequences of SEQ ID NOs:11, 12, and 13, respectively; and
   (4) a second light chain (LC2) comprising a LC2 variable region 2 (VL2), wherein the VL2 comprises a light chain complementarity determining region 1 (LCDR1), a LCDR2, and a LCDR3 having the amino acid sequences of SEQ ID NOs:14, 15, and 16, respectively;
(b) about 10 mM to about 20 mM of acetate and/or pharmaceutically acceptable acetate salt;
(c) about 6% (w/v) to about 10% (w/v) of sucrose;
(d) about 16 μg/mL to about 24 μg/mL of ethylenediaminetetraacetic acid (EDTA);
(e) about 0.01% to about 0.07% polysorbate 20; and
(f) a pH from about 4.7 to about 5.7.

In certain embodiments, the bispecific BCMA/CD3 antibody comprises a VH1 having the amino acid sequence of SEQ ID NO:7 and a VL1 having the amino acid sequence of SEQ ID NO:8. In certain embodiments, the bispecific BCMA/CD3 antibody comprises a HCl having the amino acid sequence of SEQ ID NO:9, and a LC1 having the amino acid sequence of SEQ ID NO:10. In certain embodiments, the bispecific BCMA/CD3 antibody comprises a VH2 having the amino acid sequence of SEQ ID NO:17, and a VL2 having the amino acid sequence of SEQ ID NO:18. In certain embodiments, the bispecific BCMA/CD3 antibody comprises a HC2 having the amino acid sequence of SEQ ID NO:19, and a LC2 having the amino acid sequence of SEQ ID NO:20. The bispecific BCMA/CD3 antibody can, for example, be teclistamab.

In certain embodiments, the bispecific BCMA/CD3 antibody has a concentration of about 7 mg/mL to about 13 mg/mL, about 7.5 mg/mL to about 12.5 mg/mL, about 8 mg/mL to about 12 mg/mL, or about 9 mg/mL to about 11 mg/mL. The bispecific BCMA/CD3 antibody can, for example, have a concentration of about 7 mg/mL, about 7.5 mg/mL, about 8 mg/mL, about 9 mg/mL, about 10 mg/mL, about 11 mg/mL, about 12 mg/mL, about 12.5 mg/mL, or about 13 mg/mL, or any value in between. In preferred embodiments, the bispecific BCMA/CD3 antibody has a concentration of about 10 mg/mL.

In certain embodiments, the bispecific BCMA/CD3 antibody has a concentration of about 76.5 mg/mL to about 103.5 mg/mL, about 81 mg/mL to about 99 mg/mL, about 85 mg/mL to about 95 mg/mL, or about 87 mg/mL to about 93 mg/mL. The bispecific BCMA/CD3 antibody can, for example, have a concentration of about 76.5 mg/mL, about 77 mg/mL, about 78 mg/mL, about 79 mg/mL, about 80 mg/mL, about 81 mg/mL, about 82 mg/mL, about 83 mg/mL, about 84 mg/mL, about 85 mg/mL, about 86 mg/mL, about 87 mg/mL, about 88 mg/mL, about 89 mg/mL, about 90 mg/mL, about 91 mg/ML, about 92 mg/mL, about 93 mg/mL, about 94 mg/mL, about 95 mg/mL, about 96 mg/mL, about 97 mg/mL, about 98 mg/mL, about 99 mg/mL, about 100 mg/mL, about 101 mg/mL, about 102 mg/mL, about 103 mg/mL, or about 103.5 mg/mL, or any value in between. In preferred embodiments, the bispecific BCMA/CD3 antibody has a concentration of about 90 mg/mL.

In certain embodiments, the composition comprises about 10 mM to about 20 mM, about 12 mM to about 18 mM, or about 14 mM to about 16 mM of acetate and/or a pharmaceutically acceptable acetate salt. The composition can, for example, comprise about 10 mM, about 11 mM, about 12 mM, about 13 mM, about 14 mM, about 15 mM, about 16 mM, about 17 mM, about 18 mM, about 19 mM, or about 20 mM, or any value in between of acetate and/or a pharmaceutically acceptable acetate salt. In preferred embodiments, the composition comprises about 15 mM of acetate or a pharmaceutically acceptable acetate salt.

In certain embodiments, the composition comprises about 6% (w/v) to about 10% (w/v) or about 7% (w/v) to about 9% (w/v) of sucrose. The composition can, for example comprise about 6% (w/v), about 7% (w/v), about 8% (w/v), about 9% (w/v), or about 10% (w/v), or any value in between of sucrose.

In certain embodiments, the composition comprises about 16 mg/mL to about 24 mg/mL or about 18 mg/mL to about 22 mg/mL of EDTA. The composition can, for example, comprise about 16 mg/mL, about 17 mg/mL, about 18 mg/mL, about 19 mg/mL, about 20 mg/mL, about 21 mg/mL, about 22 mg/mL, about 23 mg/mL, or about 24 mg/mL, or any value in between of EDTA. In preferred embodiments, the composition comprises about 20 mg/mL of EDTA.

In certain embodiments, the composition comprises about 0.01% to about 0.07%, about 0.02% to about 0.06%, or about 0.03% to about 0.05% of polysorbate 20 (PS 20). The composition can, for example, comprise about 0.01%, about 0.02%, about 0.03%, about 0.04%, about 0.05%, about 0.06%, or about 0.07%, or any value in between of PS 20. In preferred embodiments, the composition comprises about 0.04% PS 20.

In certain embodiments, the pH of the composition is about 4.7 to about 5.7, about 4.8 to about 5.6, about 4.9 to about 5.5. The pH of the composition can, for example, be about 4.7 about 4.8, about 4.9, about 5.0, about 5.1, about 5.2, about 5.3, about 5.4, about 5.5, about 5.6, or about 5.7, or any value in between. In preferred embodiments, the pH of the composition is about 5.2.

The stability of the presently disclosed aqueous pharmaceutical compositions, also referred to as drug product (DP), is determined based on specific amount or proportion of the BCMAxCD3 antibody and other constituents of the DP as provided herein (such as, but not limited to, acetate and/or pharmaceutically acceptable acetate salts, sucrose, PS20, and EDTA), as well as the assessment of various factors. These factors include but are not limited to the color of the solution, the pH, the turbidity, percentage of purity, number of subvisible particles, percentage of new peak(s), percentage of main component, percentage of high molecular weight species (HMWS), percentage of low molecular weight species (LMWS), percentage of sum of acidic peaks, percentage of sum of basic peaks, protein concentration, percentage of T-cell activation, and/or percentage of PS20.

Stable DP as disclosed herein should not be construed to require all the factors listed herein but rather at least one, at least two, or at least three or more of those factors. In some embodiments, the stable disclosed DP exhibits the following results for at least one, at least two, at least three or more of the factors listed in detail below herein. In the preferred embodiment, the stable DP exhibits the following results for most of the factors listed in detail below herein. In the most preferred embodiment, the stable DP exhibits the following results for all the factors listed in detail below herein.

Color of Solution

The Color of a DP solution is monitored and can be assessed to verify that the appearance of the solution is consistent with previous batches at release and over the shelf life. The color of the DP solution can reflect stability. In one embodiment, the stability of the DP is defined when having a color of solution spanning from colorless to about BY2 or less, to about BY4 or less, to about B2 or less, to about B4 or less, to about Y2 or less or to about Y4 or less as described in the European Pharmacopoeia 2.2.2, Degree of Coloration of Liquids European Pharmacopoeia (Ph. Eur.) 10th Edition monograph number 20202, July 2019.

In one embodiment, the stability is defined as having a color of solution of colorless to about BY2 or less, about B2 or less, about Y2 or less after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined as having a color of solution of colorless to about BY4 or less, to about B4 or less, to about Y4 or less after storage for about 12 months or more and at a temperature of about 5° C., after storage for about 12 months or more and at a temperature of about 25° C., and/or after storage for about 2 years or more and at a temperature of about 5° C. In the most preferred embodiment, stability is defined as having a color of solution of colorless to about BY5 or less, to about B5 or less, to about Y5 or less after storage for about 12 months or more and at a temperature of about 5° C., after storage for about 12 months or more and at a temperature of about 25° C., and/or after storage for about 2 years or more and at a temperature of about 5° C.

pH

Measuring the pH of the DP solution allows confirmation that it is consistent with previous DP batches at release and over the shelf life. In one embodiment, the stability of the DP is defined when its pH is about: 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9 or 7.0. In one embodiment, the pH of the DP is about 5.2 after storage for about 12 months or more and at a temperature of about 5° C., after storage for about 12 months or more and at a temperature of about 25° C., and/or after storage for about 2 years or more and at a temperature of about 5° C. In one embodiment, the pH ranges from about 4.7 to about 5.7 after storage for about 12 months or more and at a temperature of about 5° C., after storage for about 12 months or more and at a temperature of about 25° C., and/or after storage for about 2 years or more and at a temperature of about 5° C. In a preferred embodiment, the stability of the DP is defined when its pH ranges from about 4.8 to about 5.6 after storage for about 12 months or more and at a temperature of about 5° C., after storage for about 12 months or more and at a temperature of about 25° C., and/or after storage for about 2 years or more and at a temperature of about 5° C. In a most preferred embodiment, the stability of the DP is defined when its pH ranges from about 4.9 to about 5.5 after storage for about 12 months or more and at a temperature of about 5° C., after storage for about 12 months or more and at a temperature of about 25° C., and/or after storage for about 2 years or more and at a temperature of about 5° C.

Turbidity

Turbidity allows measuring the presence of particles in the DP solution in order to ensure consistency with previous DP batches and applicable compendia guidance at release and over the shelf life. In one embodiment, the stability of the DP is defined when its turbidity value is about: 0, 1, 2, 3, 4, 5, 6, 7, 8, 9, 10, 11, 12, 13, 14, 15, 16, 17, 18, 19, or 20 nephelometric turbidity units (NTU) after storage for about 12 months or more and at a temperature of about 5° C., after storage for about 12 months or more and at a temperature of about 25° C., and/or after storage for about 2 years or more and at a temperature of about 5° C. In one embodiment, the stability of the DP is defined when its turbidity value is about or less than 18 NTU after storage for about 12 months or more and at a temperature of about 5° C., after storage for about 12 months or more and at a temperature of about 25° C., and/or after storage for about 2 years or more and at a temperature of about 5° C. In a preferred embodiment, the stability of the DP is defined when its turbidity value is about or less than 13 NTU after storage for about 12 months or more and at a temperature of about 5° C., after storage for about 12 months or more and at a temperature of about 25° C., and/or after storage for about 2 years or more and at a temperature of about 5° C. In the most preferred embodiment, the stability of the DP is defined when its turbidity value is about or less than 8 NTU after storage for about 12 months or more and at a temperature of about 5° C., after storage for about 12 months or more and at a temperature of about 25° C., and/or after storage for about 2 years or more and at a temperature of about 5° C.

Particle Analysis

The stability of the DP is set to a specific threshold of particles contamination based on the average number of sub-visible particles. In one embodiment, the average number of particles present in the DP units tested should not exceed 100, 200, 300, 400, 500, 600, 700, 800, 900, 1000, 2000, 3000, 4000, 5000, or 6000, per container for particle size equal to 10 μm or greater. In one embodiment, the average number of particles present in the DP units tested should not exceed 6000 per container for particle size equal to 10 μm or greater. In one embodiment, the average number of particles present in the DP units tested should not exceed 100, 200, 300, 400, 500, or 600, per container for particle size equal to 25 μm or greater. In one embodiment, the average number of particles present in the DP units tested should not exceed 600 per container for particle size equal to 25 μm or greater.

cSDS Conditions

Capillary SDS-PAGE (cSDS), like gel-based SDS-PAGE, is a method for separating denatured protein based on molecular weight. This process allows quantifying DP purity and monitoring its stability at release and over the shelf life.

In one embodiment, the DP stability is defined based upon various results of cSDS variables (e.g. percent purity or presence of new peak) where the cSDS was performed under reduced or non-reduced conditions after storage for about 12 months or more and at a temperature of about 5° C., after storage for about 12 months or more and at a temperature of about 25° C., and/or after storage for about 2 years or more and at a temperature of about 5° C.

In one embodiment, the DP stability is defined as having a percent purity about: 80%, 81%, 82%, 83%, 84%, 85%, 86%, 87%, 88%, 89%, 90%, 91%, 92%, 93%, 94%, 95%, 96%, 97%, 98%, 99% or about or equal to 100% or any range in between.

In one embodiment, the DP stability is defined as showing no new peak in the cSDS results of more than 0.5%, 0.8%, 0.9%, 1.0%, 1.2%, 1.3%, 1.4%, 1.5%, 1.6%, 1.7%, 1.8%, 1.9% or more than 2% when compared to an untreated reference material.

In one embodiment, the DP stability is defined with a percent purity of about 90% or more and no new peak of more than 1.5% as compared to a reference material. In a preferred embodiment, the DP stability is defined with a percent purity of about 95% or more, and with no new peak of more than 1.2% compared to a reference material. In a most preferred embodiment, the DP stability is defined as having a percent purity of about 97% or more and with no new peak of more than 1.0% as compared to a reference material.

Size-Exclusion HPLC (SE-HPLC) Results Consistent with Stability

SE-HPLC procedure allows assessing purity of the DP and monitoring its stability under non-denaturing conditions at release and over the shelf life.

In one embodiment, the DP stability is defined based upon various results of SE-HPLC variables such as the Main Component (MC), High Molecular Weight Species (HMWS), or Low Molecular Weight Species (LMWS), after storage of the DP for about 12 months or more and at a temperature of about 5° C., after storage for about 12 months or more and at a temperature of about 25° C., and/or after storage for about 2 years or more and at a temperature of about 5° C.

In one embodiment, the DP stability is defined as having a MC of about: 80%, 81%, 82%, 83%, 84%, 85%, 86%, 87%, 88%, 89%, 90%, 91%, 92%, 93%, 94%, 95%, 96%, 97%, 98%, 99% or equal to about 100% or any range in between. In one embodiment, the DP stability is defined as having a MC of about 90% or more. In a preferred embodiment, the DP stability is defined as having a MC of about 95% or more. In the most preferred embodiment, the DP stability is defined as having a MC of about 97% or more.

In one embodiment, the DP stability is defined as having a HMWS of about: 0.1%, 0.5%, 1%, 2%, 3%, 4%, 5%, 6%, 7%, 8%, 9%, 10%, 11%, 12%, 13%, 14%, 15% or any range in between. In one embodiment, the DP stability is defined as having a HMWS of about 10% or less. In a preferred embodiment, the DP stability is defined as having a HMWS of about 5% or less. In the most preferred embodiment, the DP stability is defined as having a HMWS of about to 3% or less.

In one embodiment, the DP stability is defined as having a LMWS of about: 0.1%, 0.5%, 0.6%, 0.7%, 0.8%, 0.9%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10%. In one embodiment, the DP stability is defined as having a LMWS of about 5% or less. In a preferred embodiment, the DP stability is defined as having a LMWS of about 2% or less. In a most preferred embodiment, the DP stability is defined as having a LMWS of about 1% or less.

Capillary Isoelectric Focusing (cIEF)

The cIEF, like isoelectric gel electrophoresis (IEF) methods, separates proteins on the basis of overall charge or isoelectric point (pI). This procedure allows monitoring the distribution of charge-based isoforms of the drug product at release and over the shelf life. In one embodiment, the DP stability is defined based upon various results of cIEF variables such as the Main Peak (MP), the sum of acidic peaks or the sum of basic peaks, after DP storage for about 12 months or more and at a temperature of about 25° C., and/or after storage for about 2 years or more and at a temperature of about 5° C.

In one embodiment, the DP stability is defined as having a cIEF with a MP of about: 60%, 65%, 70%, 75%, 80%, 85%, 90%, 95%, or 100% or any range in between after DP storage for about 12 months or more and at a temperature of about 25° C., and/or after storage for about 2 years or more and at a temperature of about 5° C. In one embodiment, the DP stability is defined as having a cIEF with a MP≥60% after DP storage for about 12 months or more and at a temperature of about 25° C., and/or after storage for about 2 years or more and at a temperature of about 5° C. In a preferred embodiment, the DP stability is defined as having a cIEF with a MP≥65% after DP storage for about 12 months or more and at a temperature of about 25° C., and/or after storage for about 2 years or more and at a temperature of about 5° C. In the most preferred embodiment, the DP stability is defined as having a cIEF with a MP≥70% after DP storage for about 12 months or more and at a temperature of about 25° C., and/or after storage for about 2 years or more and at a temperature of about 5° C.

In one embodiment, the DP stability is defined as having a cIEF with a with a sum of acidic peaks totaling to about: 1%, 5%, 10%, 15%, 20%, 25%, 30%, 35%, 40%, or any range therein between after DP storage for about 12 months or more and at a temperature of about 25° C., and/or after storage for about 2 years or more and at a temperature of about 5° C. In one embodiment, the DP stability is defined as having a cIEF with a sum of acidic peaks ≤40% after DP storage for about 12 months or more and at a temperature of about 25° C., and/or after storage for about 2 years or more and at a temperature of about 5° C. In a preferred embodiment, the DP stability is defined as having a cIEF with a sum of acidic peaks totaling to about <30% after DP storage for about 12 months or more and at a temperature of about 25° C., and/or after storage for about 2 years or more and at a temperature of about 5° C. In a most preferred embodiment, the DP stability is defined as having a cIEF with a sum of acidic peaks totaling to about <25% after DP storage for about 12 months or more and at a temperature of about 25° C., and/or after storage for about 2 years or more and at a temperature of about 5° C.

In one embodiment, the DP stability is defined as having a cIEF with a sum of basic peaks totaling about: 1%, 2%, 3%, 4%, 5%, 6%, 7%, 8%, 9%, 10%, 11%, 12%, 13%, 14%, 15%, 16%, 17%, 18%, 19%, or 20%, or any range in between after DP storage for about 12 months or more and at a temperature of about 25° C., and/or after storage for about 2 years or more and at a temperature of about 5° C. In one embodiment, the DP stability is defined as having a cIEF with a sum of basic peaks totaling about 15% or less after DP storage for about 12 months or more and at a temperature of about 25° C., and/or after storage for about 2 years or more and at a temperature of about 5° C. In a preferred embodiment, the DP stability is defined as having a cIEF with a sum of basic peaks totaling less than or about 10% after DP storage for about 12 months or more and at a temperature of about 25° C., and/or after storage for about 2 years or more and at a temperature of about 5° C. In a most preferred embodiment, the DP stability is defined as having a cIEF with a sum of basic peaks totaling less than or about 8% after DP storage for about 12 months or more and at a temperature of about 25° C., and/or after storage for about 2 years or more and at a temperature of about 5° C.

Protein Concentration

Protein concentration of the DP allows verifying that it is consistent with previous DP batches at release and over the shelf life. Quantification of protein concentration can be accomplished by measuring the UV light absorbance of the drug product solution at 280 nm (A280).

10 mg/mL DP Formulation

In one embodiment, the DP stability is defined as having a protein concentration of about: 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL, or any value in between, after DP storage for about 12 months or more and at a temperature of about 25° C., and/or after storage for about 2 years or more and at a temperature of about 5° C. In one embodiment, the DP stability is defined as having a protein concentration of about 7 mg/mL to about 13 mg/mL after DP storage for about 12 months or more and at a temperature of about 25° C., and/or after storage for about 2 years or more and at a temperature of about 5° C. In a preferred embodiment, the DP stability is defined as having a protein concentration of about 8 mg/mL to about 12 mg/mL after DP storage for about 12 months or more and at a temperature of about 25° C., and/or after storage for about 2 years or more and at a temperature of about 5° C. In a most preferred embodiment, the DP stability is defined as having a protein concentration of about 9 mg/mL to 11 mg/mL after DP storage for about 12 months or more and at a temperature of about 25° C., and/or after storage for about 2 years or more and at a temperature of about 5° C.

90 mg/mL DP Formulation

In one embodiment, the DP stability is defined as having a protein concentration of about: 75 mg/mL, 76 mg/mL, 77 mg/mL, 78 mg/mL, 79 mg/mL, 80 mg/mL, 81 mg/mL, 82 mg/mL, 83 mg/mL, 84 mg/mL, 85 mg/mL, 86 mg/mL, 87 mg/mL, 88 mg/mL, 89 mg/mL, 90 mg/mL, 91 mg/mL, 92 mg/mL, 93 mg/mL, 94 mg/mL, 95 mg/mL, 96 mg/mL, 97 mg/mL, 98 mg/mL, 99 mg/mL, 100 mg/mL, 101 mg/mL, 102 mg/mL, 103 mg/mL, 104 mg/mL, or 105 mg/mL or any value in between, after DP storage for about 12 months or more and at a temperature of about 25° C., and/or after storage for about 2 years or more and at a temperature of about 5° C. In one embodiment, the DP stability is defined as having a protein concentration of about 81 mg/mL to about 99 mg/mL after DP storage for about 12 months or more and at a temperature of about 25° C., and/or after storage for about 2 years or more and at a temperature of about 5° C. In a preferred embodiment, the DP stability is defined as having a protein concentration of about 85 mg/mL to about 95 mg/mL after DP storage for about 12 months or more and at a temperature of about 25° C., and/or after storage for about 2 years or more and at a temperature of about 5° C. In a most preferred embodiment, the DP stability is defined as having a protein concentration of about 87 mg/mL to 93 mg/mL after DP storage for about 12 months or more and at a temperature of about 25° C., and/or after storage for about 2 years or more and at a temperature of about 5° C.

Peptide Mapping

Post-translational modifications (PTMs), such as oxidation, deamidation, and isomerization, are enzymatic modifications that may be detected in the structure of an antibody. In some embodiments, the PD stability is assessed based on level of PTMs in the antibody. Test articles are enzymatically digested to yield peptide segments. These peptides are then evaluated by for instance by mass spectrometry (MS), by tandem mass spectrometry (MS-MS) or Ultra High-Performance Liquid Chromatography Mass Spectroscopy (UPLC-MS). Each analyzed peptide sequence is identified relative to its known location within the overall antibody structure. Post-translational modifications are determined by comparing the measured mass of the identified peptide sequence with its expected mass.

Drug Product Potency

In vitro T-cell activation assay allows assessing the level of DP stability. This activation can be assessed by using, but not limited to, a Nuclear factor of activated T cells-Response Element (NFAT-RE)-mediated luminescence assay.

BCMAxCD3 T-Cell Activation Activity

In one embodiment, the DP stability is defined as having a BMCAxCD3-mediated T-cell activation activity, relative to a reference, of about: 40%, 50%, 60%, 70%, 80%, 90%, 100%, 110%, 120%, 130%, 140%, 150%, or 160% or any range in between after DP storage for about 12 months or more and at a temperature of about 5° C., after storage for about 12 months or more and at a temperature of about 25° C., and/or after storage for about 2 years or more and at a temperature of about 5° C. In one embodiment, the DP stability is defined as having an BMCAxCD3-mediated T-cell activation activity ranging from about 50% to about 150% relative to a reference after DP storage for about 12 months or more and at a temperature of about 25° C., and/or after storage for about 2 years or more and at a temperature of about 5° C. In a preferred embodiment, the DP stability is defined as having an BMCAxCD3-mediated T-cell activation activity ranging from about 60% to about 140% relative to a reference after DP storage for about 12 months or more and at a temperature of about 25° C., and/or after storage for about 2 years or more and at a temperature of about 5° C. In a most preferred embodiment, the DP stability is defined as having an BMCAxCD3-mediated T-cell activation activity ranging from about 80% to about 120% relative to a reference after DP storage for about 12 months or more and at a temperature of about 25° C., and/or after storage for about 2 years or more and at a temperature of about 5° C.

Polysorbate 20 (PS20)

In one embodiment, the DP stability is defined by a PS20 concentration in percentage weight to volume of about: 0.005%, 0.01%, 0.02%, 0.03%, 0.04%, 0.05%, 0.06%, 0.07%, 0.08%, 0.09%, 0.10%, or any range in between after DP storage for about 12 months or more and at a temperature of about 5° C., after storage for about 12 months or more and at a temperature of about 25° C., and/or after storage for about 2 years or more and at a temperature of about 5° C. In one embodiment, the DP stability is defined by a PS20 concentration of about 0.02% to about 0.1% after DP storage for about 12 months or more and at a temperature of about 25° C., and/or after storage for about 2 years or more and at a temperature of about 5° C. In one embodiment, the DP stability is defined by a PS20 concentration of about 0.01% to about 0.07%. In a preferred embodiment, the DP stability is defined by a PS20 concentration of about 0.02% to about 0.06% after DP storage for about 12 months or more and at a temperature of about 25° C., and/or after storage for about 2 years or more and at a temperature of about 5° C. In a most preferred embodiment, the DP stability is defined with a PS20 concentration of about 0.03% to about 0.05% after DP storage for about 12 months or more and at a temperature of about 25° C., and/or after storage for about 2 years or more and at a temperature of about 5° C.

Total Volume of the Stable Aqueous Pharmaceutical Composition

10 mg/mL Formulation

In one embodiment, the total volume of the stable aqueous pharmaceutical composition (or DP) ranges from about 5 mL to about 10 mL. In one embodiment, the total volume of the stable aqueous pharmaceutical composition (or DP) ranges from about 0.5 mL to about 20 mL, from about 1 mL to about 15 mL, from about 5 mL to about 10 mL, or from about 6 mL to about 8 mL. In one embodiment, the total volume of the stable aqueous pharmaceutical composition is about: 0.5 mL, 0.6 mL, 0.7 mL, 0.8 mL, 0.9 mL, 1 mL, 2 mL, 3 mL, 4 mL, 5 mL, 6 mL, 8 mL, 9 mL, 10 mL, 11 mL, 12 mL, 13 mL, 14 mL, 15 mL, 16 mL, 18 mL, 19 mL, 20 mL, 25 mL, or 30 mL or any range in between. In one embodiment, the total volume of the stable aqueous pharmaceutical composition is 2 mL.

90 mg/mL Formulation

In one embodiment, the total volume of the stable aqueous pharmaceutical composition (or DP) ranges from about 5 mL to about 10 mL. In one embodiment, the total volume of the stable aqueous pharmaceutical composition (or DP) ranges from about 0.5 mL to about 20 mL, from about 1 mL to about 15 mL, from about 5 mL to about 10 mL, or from about 6 mL to about 8 mL. In one embodiment, the total volume of the stable aqueous pharmaceutical composition is about: 0.5 mL, 0.6 mL, 0.7 mL, 0.8 mL, 0.9 mL, 1 mL, 2 mL, 3 mL, 4 mL, 5 mL, 6 mL, 8 mL, 9 mL, 10 mL, 11 mL, 12 mL, 13 mL, 14 mL, 15 mL, 16 mL, 18 mL, 19 mL, 20 mL, 25 mL, or 30 mL or any range in between. In one embodiment, the total volume of the stable aqueous pharmaceutical composition is 3.5 mL.

### METHODS

Provided herein is a method of treating cancer in a subject in need thereof. The method comprises administering to the subject a stable aqueous pharmaceutical composition as disclosed herein. In certain embodiments, the administration is subcutaneous.

Cancers

In some embodiments, the cancer is a hematological malignancy or a solid tumor.

In some embodiments, the hematological malignancy is a multiple myeloma, a smoldering multiple myeloma, a monoclonal gammopathy of undetermined significance (MGUS), an acute lymphoblastic leukemia (ALL), a diffuse large B-cell lymphoma (DLBCL), a Burkitt's lymphoma (BL), a follicular lymphoma (FL), a mantle-cell lymphoma (MCL), Waldenstrom's macroglobulinema, a plasma cell leukemia, a light chain amyloidosis (AL), a precursor B-cell lymphoblastic leukemia, a precursor B-cell lymphoblastic leukemia, an acute myeloid leukemia (AML), a myelodysplastic syndrome (MDS), a chronic lymphocytic leukemia (CLL), a B cell malignancy, a chronic myeloid leukemia (CML), a hairy cell leukemia (HCL), a blastic plasmacytoid dendritic cell neoplasm, Hodgkin's lymphoma, non-Hodgkin's lymphoma, a marginal zone B-cell lymphoma (MZL), a mucosa-associated lymphatic tissue lymphoma (MALT), plasma cell leukemia, anaplastic large-cell lymphoma (ALCL), leukemia or lymphoma.

In preferred embodiments, the hematological malignancy is multiple myeloma. In some embodiments, the subject has a newly diagnosed multiple myeloma. In some embodiments, the subject is relapsed or refractory to treatment with a prior anti-cancer therapeutic, such as a therapeutic used to treat multiple myeloma or other hematological malignancies.

In some embodiments, the subject is refractory or relapsed to treatment with one or more treatments or therapies, such as THALOMID® (thalidomide), REVLIMID® (lenalidomide), POMALYST® (pomalidomide), VELCADE® (bortezomib), NINLARO (ixazomib), KYPROLIS® (carfilzomib), FARADYK® (panobinostat), AREDIA® (pamidronate), ZOMETA® (zoledronic acid), DARZALEX® (daratumumab), elotozumab or melphalan, Xpovio® (Selinexor), Venclexta® (Venetoclax), GSK 916, CAR-T therapies, other BCMA-directed therapies.

Various qualitative and/or quantitative methods can be used to determine relapse or refractory nature of the disease. Symptoms that can be associated are for example a decline or plateau of the well-being of the patient or re-establishment or worsening of various symptoms associated with solid tumors, and/or the spread of cancerous cells in the body from one location to other organs, tissues or cells.

In some embodiments, the multiple myeloma is relapsed or refractory to treatment with an anti-CD38 antibody, selinexor, venetoclax, lenalinomide, bortezomib, pomalidomide, carfilzomib, elotozumab, ixazomib, melphalan or thalidomide, or any combination thereof.

In some embodiments, the multiple myeloma is a high-risk multiple myeloma. Subjects with high-risk multiple myeloma are known to relapse early and have poor prognosis and outcome. Subjects can be classified as having high-risk multiple myeloma is they have one or more of the following cytogenetic abnormalities: t(4;14)(p16;q32), t(14;16)(q32;q23), dell7p, lqAmp, t(4;14)(p16;q32) and t(14;16)(q32;q23), t(4;14)(p16;q32) and dell7p, t(14;16)(q32;q23) and dell7p, or t(4;14)(p16;q32), t(14;16)(q32;q23) and dell7p. In some embodiments, the subject having the high-risk multiple myeloma has one or more chromosomal abnormalities comprising: t(4;14)(p16;q32), t(14;16)(q32;q23), dell7p, lqAmp, t(4;14)(p16;q32) and t(14;16)(q32;q23), t(4;14)(p16;q32) and dell7p, t(14;16)(q32;q23) and dell7p; or t(4;14)(p16;q32), t(14;16)(q32;q23) and dell7p, or any combination thereof.

The cytogenetic abnormalities can be detected for example by fluorescent in situ hybridization (FISH). In chromosomal translocations, an oncogene is translocated to the IgH region on chromosome 14q32, resulting in dysregulation of these genes. t(4;14)(p16;q32) involves translocation of fibroblast growth factor receptor 3 (FGFR3) and multiple myeloma SET domain containing protein (MMSET) (also called WHSC1/NSD2), and t(14;16)(q32;q23) involves translocation of the MAF transcription factor C-MAF. Deletion of 17p (dell7p) involves loss of the p53 gene locus.

Chromosomal rearrangements can be identified using well known methods, for example fluorescent in situ hybridization, karyotyping, pulsed field gel electrophoresis, or sequencing.

Also provided herein is a method for preparing a stable aqueous pharmaceutical composition of a bispecific B-cell mature antigen (BCMA)/cluster of differentiation 3 (CD3) antibody or antigen-binding fragment thereof, the bispecific BCMA/CD3 antibody or antigen-binding fragment thereof comprising: a first heavy chain (HCl) comprising a HCl variable region 1 (VH1), wherein the VH1 comprises a heavy chain complementarity determining region 1 (HCDR1), a HCDR2, and a HCDR3 having the amino acid sequences of SEQ ID NOs:1, 2, and 3, respectively; a first light chain (LC1) comprising a LC1 variable region (VL1), wherein the VL1 comprises a light chain complementarity determining region 1 (LCDR1), a LCDR2, and a LCDR3 having the amino acid sequences of SEQ ID NOs: 4, 5, and 6, respectively; a second heavy chain (HC2) comprising a HC2 variable region 2 (VH2), wherein the VH2 comprises a heavy chain complementarity determining region 1 (HCDR1), a HCDR2, and a HCDR3 having the amino acid sequences of SEQ ID NOs:11, 12, and 13, respectively; and a second light chain (LC2) comprising a LC2 variable region 2 (VL2), wherein the VL2 comprises a light chain complementarity determining region 1 (LCDR1), a LCDR2, and a LCDR3 having the amino acid sequences of SEQ ID NOs:14, 15, and 16, respectively; the method comprising combining a composition comprising about 10 mg/mL or about 90 mg/mL of the bispecific BCMA/CD3 antibody, about 15 mM of acetate and/or a pharmaceutically acceptable acetate salt, about 8% (w/v) sucrose, about 20 mg/mL of EDTA, and about 0.04% polysorbate (PS) 20, wherein the stable aqueous pharmaceutical composition has a pH of about 5.2.

In certain embodiments, the bispecific BCMA/CD3 antibody comprises a VH1 having the amino acid sequence of SEQ ID NO:7 and a VL1 having the amino acid sequence of SEQ ID NO:8. In certain embodiments, the bispecific BCMA/CD3 antibody comprises a HCl having the amino acid sequence of SEQ ID NO:9, and a LC1 having the amino acid sequence of SEQ ID NO:10. In certain embodiments, the bispecific BCMA/CD3 antibody comprises a VH2 having the amino acid sequence of SEQ ID NO:17, and a VL2 having the amino acid sequence of SEQ ID NO:18. In certain embodiments, the bispecific BCMA/CD3 antibody comprises a HC2 having the amino acid sequence of SEQ ID NO:19, and a LC2 having the amino acid sequence of SEQ ID NO:20. The bispecific BCMA/CD3 antibody can, for example, be teclistamab.

The stable aqueous pharmaceutical compositions disclosed herein can be packaged into kits, containers, packs, dispensers, or vials.

Provided herein is a kit comprising the disclosed stable aqueous pharmaceutical and instructions for use thereof.

Also provided herein is an article of manufacture comprising a container holding the disclosed stable aqueous pharmaceutical composition. In some embodiments, the container is a vial with a stopper pierceable by a syringe.

### Embodiments

Provided here are illustrative embodiments of the disclosed technology. These embodiments are illustrative only and do not limit the scope of the present disclosure or of the claims attached hereto.

Embodiment 1 is a stable aqueous pharmaceutical composition comprising:
a) a concentration of about 7.5 mg/mL to about 12.5 mg/mL of a bispecific B-cell mature antigen (BCMA)/cluster of differentiation 3 (CD3) antibody or antigen-binding fragment thereof, the bispecific BCMA/CD3 antibody or antigen-binding fragment thereof comprising:
   (1) a first heavy chain (HCl) comprising a HCl variable region 1 (VH1), wherein the VH1 comprises a heavy chain complementarity determining region 1 (HCDR1), a HCDR2, and a HCDR3 having the amino acid sequences of SEQ ID NOs:1, 2, and 3, respectively;
   (2) a first light chain (LC1) comprising a LC1 variable region (VL1), wherein the VL1 comprises a light chain complementarity determining region 1 (LCDR1), a LCDR2, and a LCDR3 having the amino acid sequences of SEQ ID NOs: 4, 5, and 6, respectively;
   (3) a second heavy chain (HC2) comprising a HC2 variable region 2 (VH2), wherein the VH2 comprises a heavy chain complementarity determining region 1 (HCDR1), a HCDR2, and a HCDR3 having the amino acid sequences of SEQ ID NOs:11, 12, and 13, respectively; and
   (4) a second light chain (LC2) comprising a LC2 variable region 2 (VL2), wherein the VL2 comprises a light chain complementarity determining region 1 (LCDR1), a LCDR2, and a LCDR3 having the amino acid sequences of SEQ ID NOs:14, 15, and 16, respectively;
b) about 10 mM to about 20 mM of acetate and/or pharmaceutically acceptable acetate salt;
c) about 6% (w/v) to about 10% (w/v) of sucrose;
d) about 16 μg/mL to about 24 μg/mL of ethylenediaminetetraacetic acid (EDTA);
e) about 0.01% to about 0.07% polysorbate 20; and
f) a pH from about 4.7 to about 5.7.

Embodiment 1a is a stable aqueous pharmaceutical composition comprising:
a) a concentration of about 76.5 mg/mL to about 103.5 mg/mL of a bispecific B-cell mature antigen (BCMA)/cluster of differentiation 3 (CD3) antibody or antigen-binding fragment thereof, the bispecific BCMA/CD3 antibody or antigen-binding fragment thereof comprising:
   (1) a first heavy chain (HCl) comprising a HCl variable region 1 (VH1), wherein the VH1 comprises a heavy chain complementarity determining region 1 (HCDR1), a HCDR2, and a HCDR3 having the amino acid sequences of SEQ ID NOs:1, 2, and 3, respectively;
   (2) a first light chain (LC1) comprising a LC1 variable region (VL1), wherein the VL1 comprises a light chain complementarity determining region 1 (LCDR1), a LCDR2, and a LCDR3 having the amino acid sequences of SEQ ID NOs: 4, 5, and 6, respectively;
   (3) a second heavy chain (HC2) comprising a HC2 variable region 2 (VH2), wherein the VH2 comprises a heavy chain complementarity determining region 1 (HCDR1), a HCDR2, and a HCDR3 having the amino acid sequences of SEQ ID NOs:11, 12, and 13, respectively; and
   (4) a second light chain (LC2) comprising a LC2 variable region 2 (VL2), wherein the VL2 comprises a light chain complementarity determining region 1 (LCDR1), a LCDR2, and a LCDR3 having the amino acid sequences of SEQ ID NOs:14, 15, and 16, respectively;
b) about 10 mM to about 20 mM of acetate and/or pharmaceutically acceptable acetate salt;
c) about 6% (w/v) to about 10% (w/v) of sucrose;
d) about 16 μg/mL to about 24 μg/mL of ethylenediaminetetraacetic acid (EDTA);
e) about 0.01% to about 0.07% polysorbate 20; and
f) a pH from about 4.7 to about 5.7.

Embodiment 2 is the stable aqueous pharmaceutical composition of embodiment 1 or 1a, wherein the bispecific BCMA/CD3 antibody comprises a VH1 having the amino acid sequence of SEQ ID NO:7, and a VL1 having the amino acid sequence of SEQ ID NO:8.

Embodiment 3 is the stable aqueous pharmaceutical composition of embodiment 1 or 1a, wherein the bispecific BCMA/CD3 antibody comprising a HC having the amino acid sequence of SEQ ID NO:9, and a LC1 having the amino acid sequence of SEQ ID NO:10.

Embodiment 4 is the stable aqueous pharmaceutical composition of embodiment 1 or 1a, wherein the bispecific BCMA/CD3 antibody comprises a VH2 having the amino acid sequence of SEQ ID NO:17, and a VL2 having the amino acid sequence of SEQ ID NO:18.

Embodiment 5 is the stable aqueous pharmaceutical composition of embodiment 1 or 1a, wherein the bispecific BCMA/CD3 antibody comprises a HC2 having the amino acid sequence of SEQ ID NO:19, and a LC2 having the amino acid sequence of SEQ ID NO:20.

Embodiment 6 is the stable aqueous pharmaceutical composition of embodiment 1 or 1a, wherein the bispecific BCMA/CD3 antibody is teclistamab.

Embodiment 7 is the stable aqueous pharmaceutical composition of embodiment 1 or 1a, wherein the bispecific BCMA/CD3 antibody has a concentration of about 8 mg/mL to about 12 mg/mL.

Embodiment 8 is the stable aqueous pharmaceutical composition of embodiment 7, wherein the bispecific BCMA/CD3 antibody has a concentration of about 9 mg/mL to about 11 mg/mL.

Embodiment 9 is the stable aqueous pharmaceutical composition of embodiment 8, wherein the bispecific BCMA/CD3 antibody has a concentration of about 10 mg/mL.

Embodiment 10 is the stable aqueous pharmaceutical composition of embodiment 1 or 1a, wherein the bispecific BCMA/CD3 antibody has a concentration of about 85 mg/mL to about 95 mg/mL.

Embodiment 11 is the stable aqueous pharmaceutical composition of embodiment 10, wherein the bispecific BCMA/CD3 antibody has a concentration of about 87 mg/mL to about 93 mg/mL.

Embodiment 12 is the stable aqueous pharmaceutical composition of embodiment 11, wherein the bispecific BCMA/CD3 antibody has a concentration of about 90 mg/mL.

Embodiment 13 is the stable aqueous pharmaceutical composition of embodiment 1 or 1a, wherein the composition comprises about 12 mM to about 18 mM of acetate and/or pharmaceutically acceptable acetate salt.

Embodiment 14 is the stable aqueous pharmaceutical composition of embodiment 1 or 1a, wherein the composition comprises about 14 mM to about 16 mM of acetate and/or pharmaceutically acceptable acetate salt.

Embodiment 15 is the stable aqueous pharmaceutical composition of embodiment 1 or 1a, wherein the composition comprises about 15 mM of acetate and/or pharmaceutically acceptable acetate salt.

Embodiment 16 is the stable aqueous pharmaceutical composition of embodiment 1 or 1a, wherein the composition comprises about 7% (w/v) to about 9% (w/v) of sucrose.

Embodiment 17 is the stable aqueous pharmaceutical composition of embodiment 16, wherein the composition comprises about 8% (w/v) of sucrose.

Embodiment 18 is the stable aqueous pharmaceutical composition of embodiment 1 or 1a, wherein the composition comprises about 18 μg/mL to about 22 μg/mL of EDTA.

Embodiment 19 is the stable aqueous pharmaceutical composition of embodiment 1 or 1a, wherein the composition comprises about 20 μg/mL of EDTA.

Embodiment 20 is the stable aqueous pharmaceutical composition of embodiment 1 or 1a, wherein the composition comprises about 0.02% to about 0.06% of PS-20.

Embodiment 21 is the stable aqueous pharmaceutical composition of embodiment 20, wherein the composition comprises about 0.03 to about 0.05% of PS-20.

Embodiment 22 is the stable aqueous pharmaceutical composition of embodiment 21, wherein the composition comprises about 0.04% of PS-20.

Embodiment 23 is the stable aqueous pharmaceutical composition of embodiment 1 or 1a, wherein the pH is about 4.8 to about 5.6.

Embodiment 24 is the stable aqueous pharmaceutical composition of embodiment 23, wherein the pH is about 4.9 to about 5.5.

Embodiment 25 is the stable aqueous pharmaceutical composition of embodiment 24, wherein the pH is about 5.2.

Embodiment 26 is the stable aqueous pharmaceutical composition of embodiment 1 or 1a, wherein the composition comprises 10 mg/mL of the bispecific BCMA/CD3 antibody, 15 mM of acetate and/or pharmaceutically acceptable acetate salt, 8% (w/v) sucrose, 20 μg/mL of EDTA, 0.04% PS 20, and a pH of 5.2.

Embodiment 27 is the stable aqueous pharmaceutical composition of embodiment 1 or 1a, wherein the stable aqueous pharmaceutical composition is stable at a temperature of about 2-8° C. for at least two years.

Embodiment 28 is the stable aqueous pharmaceutical composition of embodiment 1 or 1a, wherein stability is defined based on color of solution, pH, turbidity, percentage of purity, percentage of new peaks, percentage of main component, percentage of high molecular weight species (HWMS), percentage of low molecular weight species (LMWS), percentage of sum of acidic peaks, percentage of sum of basic peaks, protein concentration, percentage of T cell activation, percentage of PS 20 (w/v), or any combination thereof.

Embodiment 29 is a method of treating cancer in a subject in need thereof, the method comprising administering to the subject the stable aqueous pharmaceutical composition of embodiment 1 or 1a.

Embodiment 30 is the method of embodiment 29, wherein the administering is subcutaneous.

Embodiment 31 is a method for preparing a stable aqueous pharmaceutical composition of a bispecific B-cell mature antigen (BCMA)/cluster of differentiation 3 (CD3) antibody or antigen-binding fragment thereof, the bispecific BCMA/CD3 antibody or antigen-binding fragment thereof comprising:
(1) a first heavy chain (HCl) comprising a HCl variable region 1 (VH1), wherein the VH1 comprises a heavy chain complementarity determining region 1 (HCDR1), a HCDR2, and a HCDR3 having the amino acid sequences of SEQ ID NOs:1, 2, and 3, respectively;
(2) a first light chain (LC1) comprising a LC1 variable region (VL1), wherein the VL1 comprises a light chain complementarity determining region 1 (LCDR1), a LCDR2, and a LCDR3 having the amino acid sequences of SEQ ID NOs: 4, 5, and 6, respectively;
(3) a second heavy chain (HC2) comprising a HC2 variable region 2 (VH2), wherein the VH2 comprises a heavy chain complementarity determining region 1 (HCDR1), a HCDR2, and a HCDR3 having the amino acid sequences of SEQ ID NOs:11, 12, and 13, respectively; and
(4) a second light chain (LC2) comprising a LC2 variable region 2 (VL2), wherein the VL2 comprises a light chain complementarity determining region 1 (LCDR1), a LCDR2, and a LCDR3 having the amino acid sequences of SEQ ID NOs:14, 15, and 16, respectively;
   the method comprising combining a composition comprising about 10 mg/mL of the bispecific BCMA/CD3 antibody, about 15 mM of acetate and/or a pharmaceutically acceptable acetate salt, about 8% (w/v) sucrose, about 20 mg/mL of EDTA, and about 0.04% polysorbate (PS) 20, wherein the stable aqueous pharmaceutical composition has a pH of about 5.2.

Embodiment 31a is a method for preparing a stable aqueous pharmaceutical composition of a bispecific B-cell mature antigen (BCMA)/cluster of differentiation 3 (CD3) antibody or antigen-binding fragment thereof, the bispecific BCMA/CD3 antibody or antigen-binding fragment thereof comprising:
(1) a first heavy chain (HCl) comprising a HCl variable region 1 (VH1), wherein the VH1 comprises a heavy chain complementarity determining region 1 (HCDR1), a HCDR2, and a HCDR3 having the amino acid sequences of SEQ ID NOs:1, 2, and 3, respectively;
(2) a first light chain (LC1) comprising a LC1 variable region (VL1), wherein the VL1 comprises a light chain complementarity determining region 1 (LCDR1), a LCDR2, and a LCDR3 having the amino acid sequences of SEQ ID NOs: 4, 5, and 6, respectively;
(3) a second heavy chain (HC2) comprising a HC2 variable region 2 (VH2), wherein the VH2 comprises a heavy chain complementarity determining region 1 (HCDR1), a HCDR2, and a HCDR3 having the amino acid sequences of SEQ ID NOs:11, 12, and 13, respectively; and
(4) a second light chain (LC2) comprising a LC2 variable region 2 (VL2), wherein the VL2 comprises a light chain complementarity determining region 1 (LCDR1), a LCDR2, and a LCDR3 having the amino acid sequences of SEQ ID NOs:14, 15, and 16, respectively;
   the method comprising combining a composition comprising about 90 mg/mL of the bispecific BCMA/CD3 antibody, about 15 mM of acetate and/or a pharmaceutically acceptable acetate salt, about 8% (w/v) sucrose, about 20 mg/mL of EDTA, and about 0.04% polysorbate (PS) 20, wherein the stable aqueous pharmaceutical composition has a pH of about 5.2.

Embodiment 32 is the method of embodiment 31 or 31a, wherein the bispecific BCMA/CD3 antibody comprises a VH1 having the amino acid sequence of SEQ ID NO:7, and a VL1 having the amino acid sequence of SEQ ID NO:8.

Embodiment 33 is the method of embodiment 31 or 31a, wherein the bispecific BCMA/CD3 antibody comprising a HC having the amino acid sequence of SEQ ID NO:9, and a LC1 having the amino acid sequence of SEQ ID NO:10.

Embodiment 34 is the method of embodiment 31 or 31a, wherein the bispecific BCMA/CD3 antibody comprises a VH2 having the amino acid sequence of SEQ ID NO:17, and a VL2 having the amino acid sequence of SEQ ID NO:18.

Embodiment 35 is the method of embodiment 31 or 31a, wherein the bispecific BCMA/CD3 antibody comprises a HC2 having the amino acid sequence of SEQ ID NO:19, and a LC2 having the amino acid sequence of SEQ ID NO:20.

Embodiment 36 is the method of embodiment 31 or 31a, wherein the bispecific BCMA/CD3 antibody is teclistamab.

Embodiment 37 is a kit comprising the stable aqueous pharmaceutical composition of embodiment 1 and instructions for use.

Embodiment 38 is an article of manufacture comprising a container holding a stable aqueous pharmaceutical composition of embodiment 1 or 1a.

Embodiment 39 is the article of manufacture of embodiment 38, wherein the container is a vial with a stopper pierceable by a syringe.

Embodiment 40 is the stable aqueous pharmaceutical composition of embodiment 1 or 1a for use in the treatment of cancer.

Embodiment 41 is the stable aqueous pharmaceutical composition of embodiment 1 or 1a for use in the preparation of a medicament for the treatment of cancer.

Embodiment 42 is the use of the stable aqueous pharmaceutical composition of embodiment 1 or 1a for treating in a subject in need thereof, the use comprising administering the stable aqueous pharmaceutical composition to the subject in need thereof.

Embodiment 43 is the use of embodiment 42, wherein the administration is subcutaneous.

### EXAMPLES

The following examples are provided to further describe some of the embodiments disclosed herein. The examples are intended to illustrate, not to limit, the disclosed embodiments.

### DESCRIPTION OF ANALYTICAL TESTS USED HEREIN

Analytical Tests—General Characterization

Color of Solution

Color of solution is monitored for drug product to assess appearance and ensure it is consistent with previous batches at release and over the shelf life. Color of solution may be an indicator of product stability. To determine Color of solution, test samples are visually compared to a defined set of reference solutions.

A defined volume of liquid content is transferred into a pre-scored ampoule of same dimensions as the reference solutions. Then the content of the ampoule is visually compared to European Pharmacopoeia color reference solutions. The degree of color is determined in diffuse daylight, viewed against a white background.

Color of Solution Material and Methods

Materials and methods are as described in European Pharmacopoeia 2.2.2, Degree of Coloration of Liquids European Pharmacopoeia (Ph. Eur.) 10th Edition monograph number 20202, July 2019. Briefly, test articles are compared against B (Brown), BY (Brownish-Yellow), and Y (Yellow) Color Reference Solution Sets.

Color of Solution results consistent with stability. In one embodiment, stability is defined as having a color of solution of colorless to about BY2 or less, about B2 or less, about Y2 or less after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined as having a color of solution of colorless to about BY4 or less, to about B4 or less, to about Y4 or less after storage for about 12 months or more and at a temperature of about 5° C., after storage for about 12 months or more and at a temperature of about 25° C., and/or after storage for about 2 years or more and at a temperature of about 5° C. In the most preferred embodiment, stability is defined as having a color of solution of colorless to about BY5 or less, to about B5 or less, to about Y5 or less after storage for about 12 months or more and at a temperature of about 5° C., after storage for about 12 months or more and at a temperature of about 25° C., and/or after storage for about 2 years or more and at a temperature of about 5° C.

pH

pH Materials and Methods—A daily calibrated electronic pH meter with standardized pH electrode is used to measure the pH of test articles. All calibration solutions, reference buffers, and test articles are equilibrated to, and maintained at, 25° C. prior to and during testing.

pH results consistent with stability. In one embodiment, stability is defined as having a pH range of 4.7 to about 5.7 after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined a pH range of 4.8 to about 5.6 after storage for about 12 months or more and at a temperature of about 5° C., after storage for about 12 months or more and at a temperature of about 25° C., and/or after storage for about 2 years or more and at a temperature of about 5° C. In the most preferred embodiment, stability is defined as having a pH range of 4.9 to about 5.5 after storage for about 12 months or more and at a temperature of about 5° C., after storage for about 12 months or more and at a temperature of about 25° C., and/or after storage for about 2 years or more and at a temperature of about 5° C.

Turbidity

Turbidity Materials and Methods—The materials and methods are based on European Pharmacopoeia 2.2.1, Clarity and Degree of Opalescence of Liquids.

Turbidity results consistent with stability. Test results are reported in nephelometric turbidity units (NTU). In one embodiment, stability is defined as having a turbidity value of about 18 NTU or less after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined as having a turbidity value of about 13 NTU or less after storage for about 12 months or more and at a temperature of about 5° C., after storage for about 12 months or more and at a temperature of about 25° C., and/or after storage for about 2 years or more and at a temperature of about 5° C. In the most preferred embodiment, stability is defined as having a turbidity value of about 8 NTU or less after storage for about 12 months or more and at a temperature of about 5° C., after storage for about 12 months or more and at a temperature of about 25° C., and/or after storage for about 2 years or more and at a temperature of about 5° C.

Analytical Tests—Particulate Matter

Particulate Matter (Sub-visible) Materials and Methods—All materials and methods are compliant with United States Pharmacopeia <788> Particulate Matter. A Compendial compliant Liquid Particle Counter instrument equipped with a compendial volume sampler set-up is used. Test particles are equilibrated to room temperature for at least 60 minutes, but no longer than 10 hours, prior to testing. Test particle vials are pooled in manner compliant with United States Pharmacopeia <788> Particulate Matter. As instructed by United States Pharmacopeia <788> Particulate Matter, four portions of pooled test article, each of appropriate volume, are removed and the number of particles equal to or greater than 10 μm and 25 μm are counted per portion. Results obtained for the first portion are disregarded and the remaining three results are used to calculate the mean number of particles for the preparation examined.

Particle Analysis (sub-vis) compendia compliant results—Testing results are to comply with United States Pharmacopoeia <788> Particulate Matter, European Pharmacopoeia 2.9.19, and Japanese Pharmacopoeia XVII/6.07 Particulate Contamination: Sub-visible particles. As such, the average number of particles present in the units tested should not exceed 6000 particles per container for particles size equal to 10 μm or greater and should not exceed 600 particles per container for particles size equal to 25 μm or greater.

Analytical Tests—Purity

Capillary Electrophoresis Sodium Dodecyl Sulfate (cSDS)-Reduced

cSDS Reduced Materials and Methods—Analysis employs a commercial capillary electrophoresis system with a bare fused silica capillary, 50 μm i.d.×30.2 cm length in a temperature-controlled cartridge; the capillary is equipped with a detection window transparent to ultraviolet light. The capillary is rinsed electrokinetically before each injection. The capillary is loaded with a sieving matrix consisting of an entangled polymer solution before each sample analysis. The method utilizes an SDS-MW gel migration buffer and certified protein molecular weight standards spanning a range of approximately 10 to 148 kDa. The instrument's ultraviolet absorption spectrophotometer detector is set at a wavelength of 220 nm and the capillary temperature is set to 25° C. For reducing sample treatment conditions, the test article (in duplicate) is mixed with SDS and 2-mercaptoethanol and then heated for a defined time and temperature to fully denature and reduce the protein. The reduced sample is injected electrokinetically by applying a voltage of 5 kV across the capillary for approximately 20 seconds, and then analyzed by application of a greater electric field for approximately 35 minutes. Detection is accomplished by absorbance in the far ultraviolet region of the spectrum, 220 nm. Percent of total signal data is collected for the light chain, heavy chain, and a glycosylated heavy chain (AG HC).

cSDS Reduced results consistent with stability. In one embodiment, stability is defined as having a percent purity≥90.0%, and no new peak≥1.5% compared to a validated stock of teclistamab Reference Material after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined as having a percent purity about or more than 95.0% and no new peak more than 1.2% compared to Reference Material after storage for about 12 months or more and at a temperature of about 5° C., after storage for about 12 months or more and at a temperature of about 25° C., and/or after storage for about 2 years or more and at a temperature of about 5° C. In the most preferred embodiment, stability is defined as having a percent purity about or more than 97.0% and no new peak more than 1.0% compared to Reference Material after storage for about 12 months or more and at a temperature of about 5° C., after storage for about 12 months or more and at a temperature of about 25° C., and/or after storage for about 2 years or more and at a temperature of about 5° C.

Capillary Electrophoresis Sodium Dodecyl Sulfate (cSDS)-Non-Reduced

cSDS Non-reduced Materials and Methods—Analysis employs a commercial capillary electrophoresis system with a bare fused silica capillary, 50 μm i.d.×30.2 cm length in a temperature-controlled cartridge; the capillary is equipped with a detection window transparent to ultraviolet light. The capillary is rinsed electrokinetically before each injection. The capillary is loaded with a sieving matrix consisting of an entangled polymer solution before each sample analysis. The method utilizes an SDS-MW gel migration buffer, certified protein molecular weight standards spanning a range of approximately 10 to 148 kDa, and a validated teclistamab reference material sample. The instrument's ultraviolet absorption spectrophotometer detector is set at a wavelength of 220 nm and the capillary temperature is set to 25° C. For non-reduced sample treatment conditions, the test article (in duplicate) is mixed with SDS and the alkylating reagent (N-Ethylmaleimide, to prevent disulfide bond shuffling or reformation). It is then heated for a defined time and temperature to fully denature the protein and minimize formation of fragments and artifact bands. The non-reduced sample is injected electrokinetically by applying a voltage of 5 kV across the capillary for approximately 20 seconds, and then analyzed by application of a greater electric field for approximately 35 minutes. Detection is accomplished by absorbance in the far ultraviolet region of the spectrum, 220 nm. Percent of total signal data is collected. The data is also analyzed for the presence of new peaks versus teclistamab reference material. Percent purity is defined as percent heavy chain+percent light chain.

cSDS Non-Reduced results consistent with stability. In one embodiment, stability is defined as having a percent purity of about 90.0% or more and no new peak more than 1.5% compared to Reference Material after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined as having a percent purity of about 95.0% or more and no new peak more than 1.2% compared to Reference Material after storage for about 12 months or more and at a temperature of about 5° C., after storage for about 12 months or more and at a temperature of about 25° C., and/or after storage for about 2 years or more and at a temperature of about 5° C. In the most preferred embodiment, stability is defined as having a percent purity of about 97.0% or more and no new peak more than 1.0% compared to Reference Material after storage for about 12 months or more and at a temperature of about 5° C., after storage for about 12 months or more and at a temperature of about 25° C., and/or after storage for about 2 years or more and at a temperature of about 5° C.

Size Exclusion High Performance Liquid Chromatography (SE-HPLC)

SE-HPLC Materials and Methods—Reference Material and test articles are diluted to a target protein concentration. A 20 μl volume of analyte is injected onto a 7.8 mm×30 cm size exclusion column with 5 μm particle size silica base, with a fractionation range of 10 to 500 kDa. Aqueous phosphate buffer is used as the mobile phase at a flow rate of 0.7 mL/minute and the absorbance of the eluate is monitored continuously at 280 nm. Monomer (main component or main peak), aggregates (high molecular weight species, or HMWS), and fragments (low molecular weight species, or LMWS) are separated on the column and elute at different retention times. The amounts of these species are measured by monitoring peak absorbance at 280 nm.

SE-HPLC Results Consistent with Stability

Main Component—In one embodiment, stability is defined as having a Main Component about 90.0% or more after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined as having a Main Component about 95.0% or more after storage for about 12 months or more and at a temperature of about 5° C., after storage for about 12 months or more and at a temperature of about 25° C., and/or after storage for about 2 years or more and at a temperature of about 5° C. In the most preferred embodiment, stability is defined as having a Main Component about 97.0% after storage for about 12 months or more and at a temperature of about 5° C., after storage for about 12 months or more and at a temperature of about 25° C., and/or after storage for about 2 years or more and at a temperature of about 5° C.

High Molecular Weight Species (HMWS)—In one embodiment, stability is defined as having a HMWS of about 10.0% or less after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined as having a HMWS of about 5.0% or less after storage for about 12 months or more and at a temperature of about 5° C., after storage for about 12 months or more and at a temperature of about 25° C., and/or after storage for about 2 years or more and at a temperature of about 5° C. In the most preferred embodiment, stability is defined as having a HMWS of about 3.0% or less after storage for about 12 months or more and at a temperature of about 5° C., after storage for about 12 months or more and at a temperature of about 25° C., and/or after storage for about 2 years or more and at a temperature of about 5° C.

Low Molecular Weight Species (LMWS)—In one embodiment, stability is defined as having a LMWS about 5.0% or less after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined as having a LMWS of about 2.0% or less after storage for about 12 months or more and at a temperature of about 5° C., after storage for about 12 months or more and at a temperature of about 25° C., and/or after storage for about 2 years or more and at a temperature of about 5° C. In the most preferred embodiment, stability is defined as having a LMWS about 1.0% or less after storage for about 12 months or more and at a temperature of about 5° C., after storage for about 12 months or more and at a temperature of about 25° C., and/or after storage for about 2 years or more and at a temperature of about 5° C.

Capillary Isoelectric Focusing (cIEF)

cIEF Materials and Methods—The analytical procedure is performed on a commercially available imaging cIEF analyzer equipped with an auto sampler. Analysis employs a 100-μm inner wall-coated silica capillary with an outer wall polyimide coating. In addition, an analyte solution of dilute phosphoric acid and methylcellulose, a catholyte solution of sodium hydroxide and methylcellulose, and defined type and amount of ampholytes are used. The test articles are treated with carboxypeptidase B (CPB) to remove C-terminal lysine and eliminate ambiguities introduced by the presence of multiple C-terminal variants for each charged species. The instrument's autosampler is set to 4° C. for both pre-focusing and focusing. The Pre-focusing voltage and time are 1500 V and 1 minute respectively. The Focusing voltage and time are 3000 V and 7 minutes respectively.

cIEF Results Consistent with Stability

Main Peak—In one embodiment, stability is defined as having a Main Peak≤60% after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined as having a Main Peak≤65% after storage for about 12 months or more and at a temperature of about 5° C., after storage for about 12 months or more and at a temperature of about 25° C., and/or after storage for about 2 years or more and at a temperature of about 5° C. In the most preferred embodiment, stability is defined as having a Main Peak≤70% after storage for about 12 months or more and at a temperature of about 5° C., after storage for about 12 months or more and at a temperature of about 25° C., and/or after storage for about 2 years or more and at a temperature of about 5° C.

Sum of acidic peaks—In one embodiment, stability is defined as having a Sum of acidic peaks totaling≤40% after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined as having a Sum of acidic peaks totaling≤30% after storage for about 12 months or more and at a temperature of about 5° C., after storage for about 12 months or more and at a temperature of about 25° C., and/or after storage for about 2 years or more and at a temperature of about 5° C. In the most preferred embodiment, stability is defined as having a Sum of acidic peaks totaling≤25% after storage for about 12 months or more and at a temperature of about 5° C., after storage for about 12 months or more and at a temperature of about 25° C., and/or after storage for about 2 years or more and at a temperature of about 5° C.

Sum of basic peaks—In one embodiment, stability is defined as having a Sum of basic peaks totaling about <15% after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined as having a Sum of basic peaks totaling about <10% after storage for about 12 months or more and at a temperature of about 5° C., after storage for about 12 months or more and at a temperature of about 25° C., and/or after storage for about 2 years or more and at a temperature of about 5° C. In the most preferred embodiment, stability is defined as having a Sum of basic peaks totaling about <8% after storage for about 12 months or more and at a temperature of about 5° C., after storage for about 12 months or more and at a temperature of about 25° C., and/or after storage for about 2 years or more and at a temperature of about 5° C.

Analytical Tests—Quantity

Protein Concentration by A280

Protein concentration of the drug product is determined by quantification of the absorbance at 280 nm (A280).

Protein Concentration by A280 Materials and Methods

Measurement of protein concentration is performed using a qualified and calibrated double beam UV-Vis spectrophotometer. Test articles are diluted 1:125 using 0.9% (w/v) NaCl. Samples are measured using quartz semi-micro cuvettes (1.4 ml) with a 1 cm path length and black or frosted sides. The Spectrophotometer is set to a Wavelength of 280 nm, a slit width of 1 nm, and a response of one (1) second. 0.9% (w/v) NaCl is used as the Blank control. Protein concentration (mg/mL) is calculated by dividing the product of the Test article absorbance and dilution factor by the product of the antibody's Absorptivity Constant and instrument's path length (for example, but not limited to teclistamab's Absorptivity Constant of 1.58 (mg/mL)⁻¹ cm⁻¹ and instrument's path length of 1 cm).

10 mg/mL Formulation Protein Concentration Results Consistent with Stability

In one embodiment, stability is defined as having a protein concentration of 7.5 to 12.5 mg/mL after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined as having a protein concentration of 8 to 12 mg/mL after storage for about 12 months or more and at a temperature of about 5° C., after storage for about 12 months or more and at a temperature of about 25° C., and/or after storage for about 2 years or more and at a temperature of about 5° C. In the most preferred embodiment, stability is defined as having a protein concentration of 9 mg/mL to 11 mg/mL after storage for about 12 months or more and at a temperature of about 5° C., after storage for about 12 months or more and at a temperature of about 25° C., and/or after storage for about 2 years or more and at a temperature of about 5° C.

90 mg/mL Formulation Protein Concentration Results Consistent with Stability

In one embodiment, stability is defined as having a protein concentration of 76.5 to 103.5 mg/mL after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined as having a protein concentration of 85 to 95 mg/mL after storage for about 12 months or more and at a temperature of about 5° C., after storage for about 12 months or more and at a temperature of about 25° C., and/or after storage for about 2 years or more and at a temperature of about 5° C. In the most preferred embodiment, stability is defined as having a protein concentration of 87 mg/mL to 93 mg/mL after storage for about 12 months or more and at a temperature of about 5° C., after storage for about 12 months or more and at a temperature of about 25° C., and/or after storage for about 2 years or more and at a temperature of about 5° C.

Analytical Tests—Potency

Potency (BCMAxCD3 T-cell Activity)

The in vitro T-cell activation by BCMAxCD3 is demonstrated using a Nuclear factor of activated T cells-Response Element (NFAT-RE)-mediated luminescence assay

This reporter assay uses luminescence induced by activation of the NFAT (nuclear factor of activated T cells) pathway in CD3-expressing engineered effector cells as a read-out for target cell/effector cell co-engagement and is a surrogate measure of target cell killing. Daudi B lymphoblast cells, which expresses BCMA on its cell surface, are used as the target cells. The engagement of both the anti-BCMA Fab region with the BCMA expressing target cells and the anti-CD3 Fab region with the genetically engineered CD3+ T-cells are required for T-cell activation and subsequent NFAT-RE-mediated luminescence.

BCMAxCD3 T-cell Activation Materials and Methods. Qualified teclistamab is used as Reference Material and Controls. Solutions of Jurkat TCR/CD3 effector cells at 1.0×10⁶ viable cells/mL in culture media (4% Heat-Inactivated Fetal Bovine Serum in RPMI) and a solution of Daudi B lymphoblast target cells at 4×10⁵ viable cells/mL in cell culture media are prepared. Equal volumes of effector cells and target cells are aliquoted into individual wells on a 96-well cell culture plate. The plate is then incubated at 37° C. (±2° C.) with 5% (±2%) CO₂ for 16-24 hours. After incubation, Bio-Glo™ Luciferase substrate is added to each well and agitated moderately on a plate shaker for at least 5 minutes. Within 30 minutes of the addition of the substate, luminescence (Relative Light Unit (RLU) values) is measured using a microtiter luminescence plate reader. Data is reported as percent bioactivity relative to Reference Material.

BCMAxCD3 T-Cell activation results consistent with stability. In one embodiment, stability is defined as 50%-150% bioactivity relative to Reference Material after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined 60%-140% bioactivity relative to Reference Material after storage for about 12 months or more and at a temperature of about 5° C., after storage for about 12 months or more and at a temperature of about 25° C., and/or after storage for about 2 years or more and at a temperature of about 5° C. In the most preferred embodiment, stability is defined as ranging between about 80% to 120% bioactivity relative to Reference Material after storage for about 12 months or more and at a temperature of about 5° C., after storage for about 12 months or more and at a temperature of about 25° C., and/or after storage for about 2 years or more and at a temperature of about 5° C.

Analytical Tests—Surfactant

Polysorbate-20 Quantification

Polysorbate 20 is quantitatively determined by mixed-mode ion-exchange/hydrophobic HPLC.

PS 20 Materials and Methods. Analysis conducted with a gradient HPLC equipped with a 2.1×20 mm on-line column containing a 30 μm water-wetable, mixed-mode polymeric spherical sorbent particles, an ELSD, and a temperature-controlled column compartment at 30° C. The flow rate is set to 1 mL/minute and the ELSD evaporator temperature is set to 50° C. Mobile Phase A is 2% v/v Formic acid in water and Mobile Phase B is 2% v/v Formic acid in Isopropyl alcohol. Neat Polysorbate 20 is used to create calibration and check standards. Test article samples are injected neat.

Polysorbate 20 results consistent with stability. In one embodiment, stability is defined as a PS20 concentration of 0.01-0.07% after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined as a PS 20 concentration of 0.02-0.06% after storage for about 12 months or more and at a temperature of about 5° C., after storage for about 12 months or more and at a temperature of about 25° C., and/or after storage for about 2 years or more and at a temperature of about 5° C. In the most preferred embodiment, stability is defined as a PS 20 concentration of 0.03-0.05% after storage for about 12 months or more and at a temperature of about 5° C., after storage for about 12 months or more and at a temperature of about 25° C., and/or after storage for about 2 years or more and at a temperature of about 5° C.

Analytical Tests—Routine Characterization

Peptide Map

The purpose of this test is to measure the levels of post-translational modifications, such as oxidation, deamidation, and isomerization, that may be present in the antibody structure. Test articles are enzymatically digested to yield peptide segments. These peptides are then evaluated by Ultra High-Performance Liquid Chromatography Mass Spectroscopy (UPLC-MS). Each analyzed peptide sequence is identified relative to its known location within the overall antibody structure. Post-translational modifications are determined by comparing the measured mass of the identified peptide sequence with its expected mass.

Peptide Mapping materials and methods. Samples are denatured with 6 M Guanidine, 50 mM Tris pH 8.0, 5 mM EDTA and filtered using 30 kDa centrifugal filter device (flow through discarded). The denatured samples are reduced with 1 M Dithiothreitol (DTT), followed by alkylation with 1 M sodium Iodoacetate, and further treated with DTT to quench the reaction. The reaction mixture is exchanged into digestion buffer (50 mM Tris pH 7.0, with 1 mM CaCl₂) via Sephadex G-25 columns with separate columns used for blanks, reference material, and test articles. An aliquot of 1 mg/mL Trypsin stock solution is added to the sample in digestion buffer yielding a 20 μL/mL trypsin concentration. The solution is incubated at 37° C. for 2 hours±30 minutes. The trypsinized solution is allowed to cool to room temperature and the enzyme is inactivated with Trifluoroacetic acid. The treated samples are evaluated by Ultra High-Performance Liquid Chromatography Mass Spectroscopy (UPLC-MS) equipped with a Waters Acquity BEH (Ethylene Bridged Hybrid) C18, 2.1×100 mm, 1.7 μm, 130 Å column and an attached auto sampler. Mobile phase A is 0.1% Formic Acid in water Mobile phase B is, 0.1% FA in acetonitrile (mobile phase B). The autosampler is set to 2-8° C., the column is set to 40° C. and the flow rate is set to 500 μL/minute. Eluted peptides are subjected to electrospray ionization and detected using a calibrated on-line mass spectrometry.

### Example 1: Early Formulation Screening Studies

Early formulation screening studies were designed to evaluate the impact of pH, buffer type, surfactant, and stabilizers on the chemical, physical, and biological stability of teclistamab solutions. Development work identified a broader design space for the formulation including acetate buffer within a pH range of 4.5 to 5.4 and histidine buffer within a pH range of 5.4 to 6.4. The formulation was targeted at pH 5.2 in acetate buffer and pH 6.0 in histidine buffer to have sufficient buffering capacity in the subsequent confirmatory stability study. During the initial developability evaluation of the lead BCMAxCD3 antibody, it was noted that exposure to exaggerated levels of iron correlated with increases in aggregation as measured by SEC. Therefore, EDTA was added to the formulation.

Based on the formulation screening studies, storage stability was evaluated for two formulations as listed below.
1. 10 mg/mL teclistamab in 10 mM acetate, 8% (w/v) sucrose, 20 mg/mL EDTA, 0.04% (w/v) PS 20, pH 5.2
2. 10 mg/mL teclistamab in 10 mM histidine, 8% (w/v) sucrose, 20 mg/mL EDTA, 0.04% (w/v) PS 20, pH 6.0

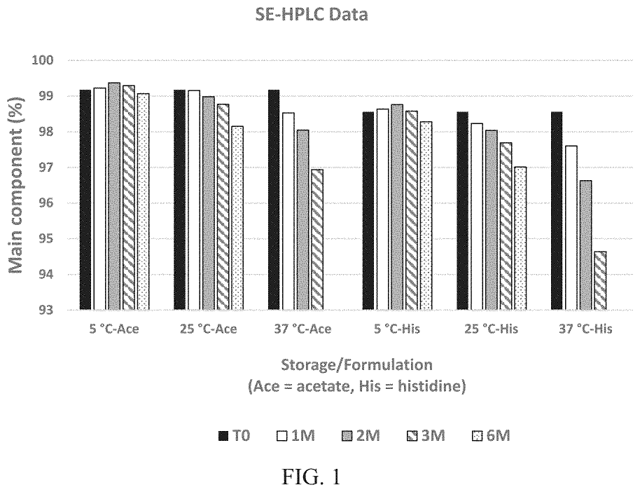

These two formulations were subjected to a storage stability study at 2-8° C., 25° C., and 37° C. Results from the study indicated no meaningful changes in stability for either formulation by cSDS (reduced and non-reduced) as well as cIEF (data not shown). Size exclusion chromatography (SE-HPLC) results indicated no loss of main component content after storage for 6 months at 2-8° C. in either formulation. However, differences were observed at elevated storage temperatures, especially at 37° C. over 3 months and at 25° C. over 6 months. The histidine buffer formulation showed greater loss of main component at 25° C. and 37° C. compared to the acetate buffer formulation (FIG. 1). These results indicated that teclistamab was more stable in the acetate buffer formulation at pH 5.2

To examine the stability of formulations with higher teclistamab concentrations, two formulations that contained 30 mg/mL and 90 mg/mL teclistamab, respectively, were placed on stability at 2-8° C. and 25° C. for up to two years. Target composition was 10 mM acetate, 8% (w/v) sucrose, 20 μg/mL EDTA disodium, 0.04% (w/v) PS 20 at pH 5.2 for both 30 and 90 mg/mL formulation. The data was compared with the 10 mg/mL formulation with the same target composition. The formulations were analyzed for main component, aggregate and fragment content by SE-HPLC, purity by cSDS (reduced and non-reduced), charge heterogeneity by cIEF, and sub-visible particulate matter by light obscuration (LO).

No substantial changes were observed for cIEF and cSDS (reduced and non-reduced) data as well as sub-visible particle counts (data not shown).

The SE-HPLC data (Table 2) shows a slight but present correlation of increased protein concentration and decreased main component. However, the magnitude of the decrease occurred over a narrow range, with all protein concentrations showing main component values consistent with the most preferred embodiment.

TABLE 2
SE-HPLC Data for 10, 30, and 90 mg/mL Formulations

| | Main component (%) | | | | | |
|---|---|---|---|---|---|---|
| Timepoint | 10 mg/mL | | 30 mg/mL | | 90 mg/mL | |
| (months) | 2-8° C. | 25° C. | 2-8° C. | 25° C. | 2-8° C. | 25° C. |
| T₀ | 99.2 | 99.2 | 99.2 | 99.2 | 98.9 | 98.9 |
| 1 | 99.2 | 99.2 | 99.0 | 98.8 | 98.6 | 98.1 |
| 2 | 99.4 | 99.0 | NA | 98.6 | NA | 97.9 |
| 3 | 99.3 | 98.8 | 99.0 | 98.6 | 98.4 | 97.9 |
| 5^{a} | 99.1 | 98.2 | 98.8 | 98.4 | 98.3 | 97.4 |
| 12 | 99.2 | NA | 98.8 | NA | 98.2 | NA |
| 18 | 99.2 | NA | 98.7 | NA | 98.1 | NA |
| 24 | 99.1 | NA | 98.7 | NA | 98.0 | NA |

^{a}10 mg/mL formulation samples pull occurred at 6 month timepoint
NA = Not applicable

At the conclusion of the Early Formulation Screening Studies, the acetate concentration was changed from 10 to 15 mM.

### Example 2: Polysorbate Concentration Range Shaking

To identify the polysorbate 20 (PS 20) levels in the acetate formulation, two shaking stress studies were performed. The first pilot study evaluated 1 mg/mL teclistamab in 10 mM acetate, 8% (w/v) sucrose, 20 μg/mL EDTA at pH 5.2 supplemented with either 0, 0.01, 0.02, 0.04, or 0.06% (w/v) PS20 against mechanical stress. The test formulations were placed on an orbital platform shaker shaken at 250 rpm for 120 hours under ambient temperature. SEC analysis of the shaken samples showed samples with no PS20 exhibited less than 30% monomer and over 70% aggregate. This demonstrates the study test conditions used produced sufficient mechanical stress to induce a catastrophic loss in DP stability. The addition of PS20 sharply increased monomer and reduced aggregate formation, with 0.02% (w/v) PS20 demonstrating monomer and aggregate levels consistent with a preferred embodiment of DP stability.

The same shaking study design was used for a second study augmented with an additional time point of 72 hours, a control sample at 0.04% (w/v) PS20 held at ambient conditions for 120 hours without shaking, and a broader, orthogonal battery of analytical assays. The second study evaluated 10 mg/mL and 90 mg/mL teclistamab in 15 mM acetate, 8% (w/v) sucrose, 20 μg/mL EDTA at pH 5.2 supplemented with either 0.02, 0.04, or 0.06% (w/v) PS20. The 10 mg/mL formulations were aliquoted into 6 mL Type 1 glass vials at a 3.5 mL fill volume and 90 mg/mL formulations were aliquoted into 2 mL Type 1 glass vials at a 2 mL fill volume.

Results are shown in Table 3 and Table 4. IE-HPLC, cSDS reduced and non-reduced, and samples after shaking exhibited essential no change in value vs. T=0 values for both the 10 mg/mL and 90 mg/mL formulations. Similarly, protein concentration, color of solution, pH, and turbidity also remained unchanged. The Sub-Vis results, when viewed in the context of the magnitude of its compendial acceptance criteria values and overall methodology, can be considered essentially the same within a given time point and verses T=0. The SEC results after shaking showed a slight trend of increases aggregation in the lowest (0.02% w/v) PS20 concentrations samples. However, these values are consistent with a preferred embodiment of DP stability.

TABLE 3
Agitation Stress Study Results for Formulations at Varying Concentrations of PS 20 - Purity and Charge Heterogeneity

| | | | | | | IE-HPLC | | | cSDS | |
|---|---|---|---|---|---|---|---|---|---|---|
| Formulation | | | SE-HPLC | | | Total | | Total | (non- | cSDS |
| Surfactant | | | Main | | | acidic | Main | basic | reduced) | (reduced) |
| Concentration ^{a} | | | component | HMWS | LMWS | groups | peak | groups | Purity | Purity |
| (w/v) | Condition | DP | (%) | (%) | (%) | (%) | (%) | (%) | (%) | (%) |
| 0.02% | | | 99.1 | 0.9 | <0.1 | 17.2 | 78.0 | 4.8 | 98.3 | 99.2 |
| 0.04% | T = 0 hr. | 10 mg/mL | 99.1 | 0.9 | <0.1 | 17.4 | 78.0 | 4.6 | 98.2 | 99.1 |
| 0.08% | | | 99.1 | 0.9 | <0.1 | 17.5 | 77.9 | 4.6 | 98.2 | 99.1 |
| 0.02% | | | 98.8 | 1.2 | <0.1 | 17.7 | 78.2 | 4.1 | 98.1 | 99.2 |
| 0.04% | T = 72 hr. | 10 mg/mL | 99.2 | 0.8 | <0.1 | 17.8 | 78.5 | 3.7 | 98.3 | 99.2 |
| 0.08% | | | 99.2 | 0.8 | <0.1 | 18.9 | 77.5 | 3.6 | 98.3 | 99.2 |
| 0.02% | | | 98.7 | 1.3 | <0.1 | 17.6 | 77.9 | 4.5 | 98.2 | 99.1 |
| 0.04% | T = 0 hr. | 90 mg/mL | 98.7 | 1.3 | <0.1 | 17.8 | 77.6 | 4.6 | 98.1 | 99.2 |
| 0.08% | | | 98.7 | 1.3 | <0.1 | 17.8 | 77.6 | 4.6 | 98.1 | 99.2 |
| 0.02% | | | 97.6 | 2.4 | <0.1 | 17.6 | 77.8 | 4.6 | 98.1 | 99.2 |
| 0.04% | T = 72 hr. | 90 mg/mL | 98.7 | 1.3 | <0.1 | 17.8 | 78.4 | 3.8 | 97.9 | 99.2 |
| 0.08% | | | 98.4 | 1.6 | <0.1 | 18.5 | 77.8 | 3.7 | 98.0 | 99.1 |

^{a} Formulation composition is 10 mg/mL or 90 mg/mL teclistamab in 15 mM acetate, 8% (w/v) sucrose, 20 μg/mL EDTA disodium, pH 5.2 in addition to PS 20

TABLE 4
Agitation Stress Study Results for 10 mg/mL and 90 mg/mL
DP Formulations at Varying Concentrations of PS 20

| Formulation | | | Sub-Visible Particulate | | |
|---|---|---|---|---|---|
| Surfactant | | | Matter LO | | |
| Concen- | | | ≥10 μm | ≥25 μm | |
| tration^{a} | | | Particles/ | Particles/ | Potency |
| (w/v) | Timepoint | DP | container | container | (%) |
| 0.02% | | | 53 | 1 | 103 |
| 0.04% | T = 0 hr. | 10 mg/mL | 126 | 5 | 88 |
| 0.08% | | | 64 | 4 | 97 |
| 0.02% | | | 191 | 32 | 97 |
| 0.04% | T = 120 hr. | 10 mg/mL | 308 | 9 | 106 |
| 0.08% | | | 225 | 5 | 112 |
| 0.02% | | | 15 | 4 | 109 |
| 0.04% | T = 0 hr. | 90 mg/mL | 31 | 6 | 107 |
| 0.08% | | | 12 | 3 | 102 |
| 0.02% | | | 31 | 9 | 99 |
| 0.04% | T = 120 hr. | 90 mg/mL | 36 | 5 | 91 |
| 0.08% | | | 91 | 41 | 97 |

^{a}Formulation composition is 10 mg/mL or 90 mg/mL teclistamab in 15 mM acetate, 8% (w/v) sucrose, 20 μg/mL EDTA disodium, pH 5.2 in addition to PS 20

Taken together, these studies demonstrate 0.02-0.08% (w/v) PS20 protects teclistamab from mechanical stress induced instability

### Example 3: Freeze Thaw Stress Study

Studies were conducted to demonstrate the robustness of the formulation against physicochemical stresses induced by repeated freeze-thaw cycles.

In this study, 10 mg/mL and 90 mg/mL formulations (15 mM acetate, 8% (w/v) sucrose, 20 μg/mL EDTA, 0.04% (w/v) PS20, pH 5.2) were aliquoted into 5 mL polycarbonate containers and placed upright in a −70° C. freezer. After freezing was complete, vials were removed from the freezer and placed at ambient temperature for thawing. Once fully thawed, the containers were gently swirled to ensure solution homogeneity and then additional freezing thawing (F/T) cycles were repeated until a total of 5 cycles were completed. Control samples did not undergo any freeze-thaw cycles. Both control and F/T samples were analyzed.

As presented in Table 5, quality attributes for samples that experienced five F/T cycles were nearly identical to the control samples that were stored at 2-8° C. and not subjected to the freeze-thaw conditions. Additionally, no significant changes in protein concentration, color of solution, pH, or turbidity were observed.

TABLE 5
Impact of repeated freeze-thaw cycles on 10
mg/mL and 90 mg/mL DP quality attributes

| | | 10 mg/mL | | 90 mg/mL | |
|---|---|---|---|---|---|
| | | | 5X | | 5X |
| Assay | Attributes | Control | F/T | Control | F/T |
| Particulate | 10 μm Particles/ | 100 | 55 | 23 | 21 |
| Matter | container | | | | |
| (sub-vis) | 25 μm Particles/ | 4 | 0 | 3 | 3 |
| | container | | | | |
| Bioassay | % Bioactivity | 96 | 110 | 103 | 97 |
| SE-HPLC | % Aggregate | 0.9 | 0.8 | 1.2 | 1.2 |
| | % Monomer | 99.1 | 99.2 | 98.8 | 98.8 |
| | % Fragment | 0.0 | 0.0 | 0.0 | 0.0 |
| IE-HPLC | % Total acidic species | 17.8 | 17.7 | 17.7 | 17.7 |
| | % Main peak | 77.6 | 78.0 | 77.9 | 77.9 |
| | % Total basic species | 4.5 | 4.3 | 4.4 | 4.3 |
| cSDS (non- | % Purity | 98.3 | 98.2 | 97.8 | 97.9 |
| reduced) | | | | | |
| cSDS (reduced) | % Purity | 99.1 | 99.0 | 99.0 | 99.0 |

The results of this study demonstrates the robustness of the formulation at 10 mg/mL and 90 mg/mL against freeze-thaw induced stress.

### Example 4: Metal Spiking Study

A study was performed to evaluate the impact of metal ions potentially present or introduced in the Drug Product during manufacturing processes.

Test formulations consisted of 10 mg/mL or 90 mg/mL teclistamab, 15 mM acetate, 8% sucrose, 20 μg/mL EDTA, and 0.04% PS 20 at pH 5.2 with or without the addition of Iron (Fe3+), Chromium (Cr3+), Copper (Cu2+), Nickel (Ni2+), and Molybdenum (Mo5+). The selection of metals is based on the composition of metal alloy components potentially present in manufacturing processes. To demonstrate robustness, exaggerated stress conditions were created by utilizing metal concentrations two to four times greater than the highest values that are seen in commercial GMP Drug Product manufacturing processes.

The 10 mg/mL test formulations were aliquoted into 6R vials at a fill volume of 3.5 mL and 90 mg/mL test formulations were aliquoted into 2 mL vials at a fill volume of 2.0 mL. All vials were stoppered, capped, and crimp sealed. The vials were placed on stability at recommended (5° C.) and accelerated (25° C.) conditions for up to 6 months. At designated time points, samples were pulled and assayed.

As shown in Table 6, quality attributes for the 10 mg/mL and 90 mg/mL formulations stressed with metal ions after 6 months of storage at 2-8° C. were nearly identical to their respective control formulations. Further there was little to no change in quality attribute values at T=6 months compared to the T=0 values for all test formulations stored at 2-8° C. No meaningful changes in protein concentration, color of solution, pH, or turbidity were observed for any formulations tested over the storage time. Lastly, the quality attribute values of all metal spiked formulations and stored at 2-8° C. for six months were consistent with the most preferred embodiment of stability.

As shown in Table 7, quality attributes for the 10 mg/mL and 90 mg/mL formulations stressed with metal ions after 6 months of storage at 25° C. were nearly identical to their respective control formulations. A slight to minimal change in some quality attribute values was seen at T=6 months as compared to the T=0 values for all test formulations. The degree of change was consistent with the expected degradation of those monoclonal antibodies quality attributes when at stored for six months at 25° C. No meaningful changes in protein concentration, color of solution, pH, or turbidity were observed for any formulations tested over storage time. Lastly, the quality attribute values of all metal spiked formulations and stored at 25° C. for six months were consistent with the preferred embodiment of stability.

Taken together, the data demonstrates the robustness of the formulation at 10 mg/mL and 90 mg/mL against potential oxidative stresses experienced during normal and/or exaggerated Drug Product manufacturing processes and storage conditions.

TABLE 6
Impact of Metal Ions on 10 mg/mL and 90 mg/mL
DP Stability for 6 months at 2-8° C.

| | | Formulation | | | | | | | |
|---|---|---|---|---|---|---|---|---|---|
| | | | | 10 mg/mL + | | | | 90 mg/mL + | |
| | | 10 mg/mL | | metals | | 90 mg/mL | | metals | |
| Assay | Attribute | T = 0 | 6 m | T = 0 | 6 m | T = 0 | 6 m | T = 0 | 6 m |
| Particulate | 10 μm | 36 | 19 | 67 | 27 | 88 | 16 | 594 | 179 |
| Matter | Particles/container | | | | | | | | |
| (sub-vis) | 25 μm | 15 | 1 | 14 | 5 | 61 | 1 | 347 | 134 |
| | Particles/container | | | | | | | | |
| Bioassay | % Bioactivity | 101 | 95 | 104 | 98 | 94 | 92 | 115 | 96 |
| SE-HPLC | % Aggregate | 0.8 | 0.7 | 0.8 | 0.8 | 1.2 | 1.6 | 1.3 | 1.7 |
| | % Monomer | 99.2 | 99.3 | 99.2 | 99.2 | 98.7 | 98.4 | 98.7 | 98.3 |
| | % Fragment | <0.1 | <0.1 | <0.1 | <0.1 | <0.1 | <0.1 | <0.1 | <0.1 |
| IE-HPLC | % Total | 18.6 | 19.6 | 19.3 | 19.7 | 18.6 | 19.4 | 18.6 | 19.6 |
| | acidic species | | | | | | | | |
| | % Main peak | 78.3 | 77.1 | 77.3 | 76.9 | 78.1 | 77.1 | 78 | 76.7 |
| | % Total | 3.1 | 3.3 | 3.4 | 3.4 | 3.4 | 3.6 | 3.4 | 3.7 |
| | basic species | | | | | | | | |
| Subunit | Total BCMA | 3.1 | 3.2 | 3.1 | 3.4 | 3.4 | 3.4 | 3.4 | 3.4 |
| LCMS | scFc oxidation | | | | | | | | |
| | Total CD3 | 2.5 | 3 | 2.7 | 3.1 | 2.7 | 3.2 | 2.5 | 3.2 |
| | scFc oxidation | | | | | | | | |
| cSDS (non- | % Purity | 98.9 | 98.4 | 98.8 | 98.7 | 98.8 | 98.4 | 98.7 | 97.7 |
| reduced) | | | | | | | | | |
| cSDS | % Purity | 98.9 | 98.8 | 98.9 | 98.7 | 98.8 | 98 | 98.7 | 97.8 |
| (reduced) | | | | | | | | | |

TABLE 7
Impact of Metal Ions on 10 mg/mL and 90 mg/mL DP Stability for 6 months at 25° C.

| | | Formulation | | | | | | | |
|---|---|---|---|---|---|---|---|---|---|
| | | | | 10 mg/mL + | | | | 90 mg/mL + | |
| | | 10 mg/mL | | metals | | 90 mg/mL | | metals | |
| Assay | Attribute | T = 0 | 6 m | T = 0 | 6 m | T = 0 | 6 m | T = 0 | 6 m |
| Particulate | ³10 mm | 36 | 7 | 67 | 60 | 88 | 3 | 594 | 10 |
| Matter | Particles/container | | | | | | | | |
| (sub-vis) | ³25 mm | 15 | 0 | 14 | 9 | 61 | 1 | 347 | 1 |
| | Particles/container | | | | | | | | |
| Bioassay | % Bioactivity | 101 | 51 | 104 | 58 | 94 | 63 | 115 | 58 |
| SE-HPLC | % Aggregate | 0.8 | 1 | 0.8 | 1.2 | 1.2 | 2.3 | 1.3 | 2.6 |
| | % Monomer | 99.2 | 99 | 99.2 | 98.7 | 98.7 | 97.6 | 98.7 | 97.4 |
| | % Fragment | <0.1 | <0.1 | <0.1 | <0.1 | <0.1 | <0.1 | <0.1 | <0.1 |
| IE-HPLC | % Total | 18.6 | 26.1 | 19.3 | 28 | 18.6 | 24.1 | 18.6 | 24.6 |
| | acidic species | | | | | | | | |
| | % Main peak | 78.3 | 69.7 | 77.3 | 67.2 | 78.1 | 70.9 | 78 | 70.3 |
| | % Total | 3.1 | 4.3 | 3.4 | 4.8 | 3.4 | 5 | 3.4 | 5.1 |
| | basic species | | | | | | | | |
| Subunit | Total BCMA | 3.1 | 4.9 | 3.1 | 4.1 | 3.4 | 4.1 | 3.4 | 4.3 |
| LCMS | scFc oxidation | | | | | | | | |
| | Total CD3 | 2.5 | 4.7 | 2.7 | 4.1 | 2.7 | 4 | 2.5 | 4.1 |
| | scFc oxidation | | | | | | | | |
| cSDS (non- | % Purity | 98.9 | 97.1 | 98.8 | 97.2 | 98.8 | 96.6 | 98.7 | 96.9 |
| reduced) | | | | | | | | | |
| cSDS | % Purity | 98.8 | 97.7 | 98.7 | 97.5 | 98.8 | 98 | 98.7 | 97.8 |
| (reduced) | | | | | | | | | |

### Example 5: Formulation Robustness Development

Study Design

A stability monoclonal antibody formulation is the result of the net effect of the complex interactions between the formulation's components. To evaluate these interactions, Design of Experiments (DOE) methodology was used to create a multi-factorial stability study in which a statistically determined number of experimental test formulations are generated that simultaneously vary the parameters of formulation components. Statistical analysis of the study results provides a quantitative understanding of how formulation parameters interact to impact stability attributes and, in turn, demonstrate the robustness of formulation. Experimental test formulations of BCMA x CD3 drug product were held at recommended (5° C.) and accelerated (25° C.) conditions for up to 12 months and six months, respectively. The formulation components evaluated were protein concentration, acetate concentration, sucrose concentration, polysorbate 20 concentration, EDTA concentration, and pH. The ranges of the factor concentrations tested are listed in Table 8 (target formulation values included in table to provide context). The study encompassed both 10 mg/mL and 90 mg/mL DP formulations. A typical protein concentration range for evaluation is +/−12% of the target value. Therefore, the protein concentration values selected for the study represent the 88% of the 10 mg/mL and 111% of the of the 90 mg/mL formulations.

TABLE 8
Ranges of the factor concentrations tested

| Test Factors | Low | Target | High |
|---|---|---|---|
| Protein (mg/mL) | 8.8 | n/a | 100 |
| pH | 4.6 | 5.2 | 5.8 |
| Acetate (mM) | 10 | 15 | 20 |
| Sucrose concentration (% w/v) | 6 | 8 | 10 |
| EDTA concentration (μg/mL) | 7.5 | 20 | 32.5 |
| PS20 concentration (% w/v) | 0.015 | 0.04 | 0.065 |

Based on this criterion, JMP® statistical software was used to create a Fractional Factorial Design (Table 9).

TABLE 9
Compositions of Study Test Formulations

| Study Test | Teclistamab | | | | | |
|---|---|---|---|---|---|---|
| Formulation | (mg/mL) | Acetate | pH | Sucrose | EDTA | PS20 |
| 1 | 100 | 10 | 5.8 | 10 | 0.015 | 7.5 |
| 2 | 100 | 10 | 4.6 | 6 | 0.065 | 7.5 |
| 3 | 100 | 20 | 5.8 | 6 | 0.065 | 7.5 |
| 4 | 100 | 10 | 5.8 | 6 | 0.015 | 32.5 |
| 5 | 8.8 | 20 | 5.8 | 10 | 0.015 | 32.5 |
| 6 | 8.8 | 10 | 4.6 | 10 | 0.015 | 32.5 |
| 7 | 100 | 10 | 4.6 | 10 | 0.065 | 32.5 |
| 8 | 100 | 20 | 5.8 | 10 | 0.065 | 32.5 |
| 9 | 8.8 | 20 | 5.8 | 6 | 0.015 | 7.5 |
| 10 | 8.8 | 10 | 5.8 | 10 | 0.065 | 7.5 |
| 11 | 100 | 20 | 4.6 | 10 | 0.015 | 7.5 |
| 12 | 8.8 | 10 | 5.8 | 6 | 0.065 | 32.5 |
| 13 | 8.8 | 20 | 4.6 | 10 | 0.065 | 7.5 |
| 14 | 8.8 | 10 | 4.6 | 6 | 0.015 | 7.5 |
| 15 | 100 | 20 | 4.6 | 6 | 0.015 | 32.5 |
| 16 | 8.8 | 20 | 4.6 | 6 | 0.065 | 32.5 |

The 16 formulations were filled into the 6R vials at 2 mL fill volume per vial (highest vial size and lowest fill volume among 10 and 90 mg/mL DP presentation) to reflect worst-case headspace. Table 10 shows the overall ranges of formulation parameters tested. Measured acetate concentrations after UF/DF during sample preparation were higher than nominal levels before UF/DF due to Gibbs-Donnan effect limitations.

TABLE 10
Ranges of formulation parameters tested

| | Lowest level | Highest level |
|---|---|---|
| Formulation parameter | tested | tested |
| JNJ-64007957 concentration (mg/mL) | 8.2 | 107.5 |
| pH | 4.5 | 5.9 |
| Acetate concentration (mM) | 10 | 41 |
| Sucrose concentration (% w/v) | 5.9 | 10.9 |
| EDTA concentration (mg/mL) | 4.7 | 34.8 |
| Polysorbate 20 concentration (% w/v) | 0.012 | 0.058 |

The vials were stoppered, capped, and crimp sealed. The vials were placed on stability at recommended (5° C.) and accelerated (25° C.) conditions. At designated time points, samples were pulled and assayed.

Study Results

The test results for each attribute of the sixteen formulations at study initiation (time zero), after 6 months at accelerated temperature (25° C.) and after 6 and 12 months at the recommended storage condition (5° C.) are presented in Table 11. The data are reported as the range, mean, and standard deviation of the eight formulations for each attribute.

The analytical results for all formulations held for 6 and 12 months at 5° C. demonstrated little change in the assay test values indicating stability. The ability for all formulations with multi-variant ranges in excipient concentrations to yield a narrow range of assay test result values for all but one assay (turbidity) demonstrates the robustness of the formulation within the boundaries and storage conditions tested. Additionally, the full range of values observed for all but one assay in this study were consistent with when held at 2-8° C.

Regarding turbidity, the calculated average turbidity value for all formulations held for 12 months at 5° C. (6.4 NTU) and was consistent with the definition of a stable aqueous pharmaceutical composition as provided for herein. However, the range of assay values observed and the calculated standard deviation was relatively larger than those observed for the other the assays. A further analysis of the data showed 3 of the 16 test formulation consistently reported a turbidity value above 15 NTU, but no more than 20 NTU, at all time points and temperatures. These formulations were three of the four test formulation in the study formulated with a high pH value (5.8) and high protein concentration (100 mg/mL). When the turbidity values for the four high pH value (5.8) and high protein concentration (100 mg/mL) test formulations are omitted, the calculated average, standard deviation, and range of the turbidity assay values for the remaining 12 formulations held for 12 months at 5° C. were 3.5 NTU, 1.6 NTU, and 1.4 to 6.3 NTU, respectively, and consistent with the most preferred embodiment of stability.

The analytical results for all formulations held for 6 months at accelerated (25° C.) storage conditions showed degradation effects consistent with the stability profile of BCMAxCD3 antibody exposed to prolonged accelerated storage conditions as demonstrated by the increase in the magnitude of the range of result values. However, the calculated average value for 7 of the 9 assay results were consistent with the most preferred embodiment of stability when held for six months at accelerated (25° C.) storage conditions.

TABLE 11
Summary of Study Data (Range, Mean, and Standard Deviation)

| | | Time and Storage Conditions | | | | | | | | | | | |
|---|---|---|---|---|---|---|---|---|---|---|---|---|---|
| | | T = 0M | | | 6M 25° C. | | | 6M 5° C. | | | 12M 5° C. | | |
| Assay | | Range | Mean | SD | Range | Mean | SD | Range | Mean | SD | Range | Mean | SD |
| cIEF | % area | 78.4-78.8 | 78.6 | 0.1 | 64.3-73.1 | 69.9 | 3.0 | 78.1-78.6 | 78.4 | 0.2 | 77.1-78.1 | 77.6 | 0.4 |
| | Main Peak | | | | | | | | | | | | |
| | % Sum of | 17.7-18.0 | 17.8 | 0.1 | 21.0-31.0 | 24.7 | 3.6 | 17.8-18.5 | 18.1 | 0.2 | 18.3-19.5 | 18.8 | 0.4 |
| | Acidic peaks | | | | | | | | | | | | |
| | % Sum of | 3.5-3.9 | 3.6 | 0.1 | 4.5-6.7 | 5.5 | 0.8 | 3.3-3.7 | 3.5 | 0.1 | 3.4-3.8 | 3.6 | 0.2 |
| | Basic peaks | | | | | | | | | | | | |
| cSDS | % Purity | 97.5-99.0 | 98.2 | 0.3 | 96.5-98.4 | 97.8 | 0.4 | 97.9-98.8 | 98.4 | 0.3 | 98.4-98.9 | 98.7 | 0.2 |
| | (non-reduced) | | | | | | | | | | | | |
| | % Purity | 97.5-99.0 | 98.8 | 0.4 | 97.5-98.1 | 97.8 | 0.2 | 99.1-99.8 | 98.9 | 0.1 | 98.7-98.7 | 98.5 | 0.1 |
| | (reduced) | | | | | | | | | | | | |
| SE- | % | 0.9-1.8 | 1.3 | 0.3 | 0.7-3.3 | 2.0 | 0.9 | 0.7-2.3 | 1.4 | 0.6 | 0.7-2.4 | 1.4 | 0.6 |
| HPLC | Aggregate | | | | | | | | | | | | |
| | % | 98.2-99.1 | 98.7 | 0.3 | 96.7-99.2 | 98.0 | 0.9 | 97.7-99.3 | 98.6 | 0.6 | 97.5-99.3 | 98.6 | 0.6 |
| | Monomer | | | | | | | | | | | | |
| | % | n/a | <0.1 | 16 of 16 | <0.1-0.1 | n/a | 10 of 16 | n/a | <0.1 | 16 of 16 | n/a | <0.1 | 16 of 16 |
| | Fragment | | | <0.1 | | | <0.1 | | | <0.1 | | | <0.1 |
| Turbidity | NTU | 1.4-17.8 | 6.1 | 5.7 | 1.4-19.5 | 6.3 | 6.3 | 1.5-18.6 | 6.2 | 5.8 | 1.6-18.8 | 6.4 | 5.7 |

Statistical Analysis of Study Results

A general linear model was used to statistically analysis the 12 Month 2-8° C. data to determine which factors had a statistically significant effect verse a given response. The analysis was conducted with the actual measured values for each factor in each test formulation after UF/DF processing. Table 12 lists the calculated p-values for each factor verses a given response and corresponding adjusted R-squared values generated by the model. p-values less than 0.05 are considered statistically significant.

TABLE 12
calculated p-values for each factor verses a given response

| | P-Value | | | | | | | |
|---|---|---|---|---|---|---|---|---|
| | Monomer | Aggregate | cSDSr | cSDSnr | Acid | Main | Basic | Turbidity |
| Factor | (%) | (%) | (%) | (%) | (%) | (%) | (%) | (NTU) |
| API | 0.00 | 0.00 | 0.52 | 0.37 | 0.02 | 0.74 | 0.00 | 0.07 |
| Acetate | 0.04 | 0.04 | 0.53 | 0.98 | 0.80 | 0.57 | 0.37 | 0.61 |
| (mM) | | | | | | | | |
| pH | 0.00 | 0.00 | 0.01 | 0.26 | 0.00 | 0.00 | 0.25 | 0.03 |
| Sucrose | 0.36 | 0.44 | 0.08 | 0.64 | 0.37 | 0.22 | 0.11 | 0.47 |
| (% w/v) | | | | | | | | |
| EDTA | 0.41 | 0.33 | 0.69 | 0.87 | 0.88 | 0.20 | 0.12 | 0.29 |
| (mg/mL) | | | | | | | | |
| PS 20 | 0.37 | 0.50 | 0.91 | 0.77 | 0.54 | 0.88 | 0.71 | 0.34 |
| (% w/v) | | | | | | | | |
| R-sq(adj) | 93.18% | 92.22% | 63.56% | 0.00% | 86.02% | 85.28% | 83.63% | 54.28% |

API Concentration

API concentration showed a statistically significant negative correlation with % monomer while also showing a corresponding, statistically significant positive correlation with % aggregate. These trends indicate that increased API concentration correlates with decreased stability as measured by SEC. However, within the concentration range evaluated in this study (8-100 mg/mL), the range of % monomer and % aggregate assay results observed were consistent with the definition of a stable aqueous pharmaceutical composition as provided for herein.

API concentration also showed a statistically significant negative correlation with % acid peak while also showing a statistically significant positive correlation with % basic peak. However, as shown in Table 12, the range of result values for % acid peak and % basic peak are considered extremely narrow for those two attributes. Therefore, while calculated to be of statistical significance, API concentration from 8-100 mg/mL is not considered to have a practical impact on the % acid and % basic peaks.

Formulation pH

Formulation pH showed a statistically significant negative correlation with % monomer while also showing a corresponding, statistically significant positive correlation with % aggregate. These trends indicate that increased pH correlates with decreased stability as measured by SEC. However, within the pH range evaluated in this study (4.6-5.8), the range of % monomer and % aggregate assay results observed were consistent with the most preferred embodiment of stability.

Formulation pH showed a statistically significant positive correlation with turbidity indicating that increased pH correlates with decreased stability as measured by turbidity. However, the r-squared (adj) value for the linear model was relatively low (54.28%). This is possibly related to the observations above in which turbidity values greater than 15 NTU were consistently observed in three test formulations formulated at high pH value (5.8) and high protein concentration (100 mg/mL). The three turbidity result values may in turn be skewing the linear model used of this analysis. However, within the pH range evaluated in this study (4.6-5.8), the range of turbidity results for low protein concentration test formulations observed were consistent with the most preferred embodiment of stability.

pH also showed a statistically significant negative correlation with % main peak and % purity by cSDS-reduced while also showing a statistically significant positive correlation with % basic peak. However, also similar to API concentration, and as shown in Table 12, the range of result values for % acid peak, % main peak and % purity by cSDS-reduced are considered extremely narrow for those three attributes. Therefore, while calculated to be of statistical significance, pH from 4.6 to 5.8 is not considered to have a practical impact on the % acid, % basic peak, and % purity by cSDS-reduced.

Acetate Concentration

Acetate Formulation showed a statistically significant negative correlation with % monomer while also showing a corresponding, statistically significant positive correlation with % aggregate. These trends indicate that increased acetate concentration correlates with decreased stability as measured by SEC. However, within the acetate concentration range evaluated in this study (10-20 mM), the range of % monomer and % aggregate assay results observed were consistent with the most preferred embodiment of stability.

### Example 6: Formulated Drug Bulk Production

Process Description

Processing Solutions

TABLE 13
Processing Solutions Target Composition and Ranges

| Solution | Composition and Ranges |
|---|---|
| Diafiltration Buffer | 10 mM Acetate, 8.6% Sucrose (w/v), pH 4.9 |
| Polysorbate 20, EDTA | 10 mM Acetate, 8% (w/v) Sucrose; 4% (w/v) |
| Stock Solution | PS20; 2 g/L EDTA, pH 5.2 |

Ultrafiltration/diafiltration (UF/DF)

Ultrafiltration/diafiltration (UF/DF) was performed to re-formulate the teclistamab Planova filtrate intermediate manufacturing solution to a pre-formulated bulk (pFB) solution consisting of 90 mg/mL teclistamab, 10 mM Acetate, 8% (w/v) Sucrose, pH 4.9.

### Preparation of Teclistamab Formulated Bulk (FB)

Polysorbate 20 (4.0% w/v) and EDTA (2 mg/mL) stock solution was added to the pFB at a 1:100 dilution to obtain a final concentration of 0.04% (w/v) Polysorbate 20, and 20 μg/mL EDTA yielding the Formulated Bulk (FB) consisting of 90 mg/mL teclistamab in 15 mM Acetate, 8% (w/v) Sucrose, 0.04% Polysorbate 20, 20 μg/mL EDTA, pH 5.2. The FB solution was then mixed uniformly. Final filtration of the Formulated Bulk was achieved using a sterile 0.45/0.22 μm filter immediately followed with a subsequent, in-line 0.22 μm filter.

Final Bulk Fill

Following final filtration, the FB was filled into polycarbonate Biotainer(s). The fill volume is 20% to 90% of the biotainer's stated volume.

Final Bulk Storage and Shipping

Storage and Shipment Conditions of the Formulated Bulk Prior to Drug Product production was 5° C.±3C protected from light if FB was stored for about one week or less or −40° C.±10° C. protected from light if FB was stored for more than one week.

### Example 7: 90 mg/mL Drug Product Formulation: Composition and Components of Primary Packaging

Provided herein is a tabular summary of the composition of the 90 mg/mL Teclistamab Drug Product Formulation in Table 14.

TABLE 14
Composition of 90 mg/mL Teclistamab Drug Product

| Component | Amount per mL | |
|---|---|---|
| Teclistamab | 90 | mg |
| Sodium acetate trihydrate | 1.497 | mg |
| Glacial acetic acid | 0.240 | mg |
| Sucrose | 8 | mg |
| Polysorbate 20 | 0.40 | mg |
| EDTA Disodium salt, Dihydrate | 0.02 | mg |

| | |
|---|---|
| Water for Injection | q.s to 1.0 mL |

The 90 mg/mL Teclistamab drug product (DP) primary packaging consists of a glass vial, a polymer vial stopper, and an aluminum seal. Table 15 provides the specific components for the primary packaging material of the 90 mg/mL DP presentation.

TABLE 15
90 mg/mL DP Primary packaging material components

| Component | Description |
|---|---|
| Glass vial | 2 mL glass Type 1 borosilicate |
| Stopper | 13 mm Stopper 4432/50 Gray with Flurotec Coating, |
| | RTS |
| Seals | 13 mm silver aluminum seal with orange colored |
| | flip-off cap |

### Example 8: Omg/mL DP Solution Production

Dilution of Teclistamab Formulated Bulk (FB)

Dilution was performed to re-formulate the teclistamab Formulated Bulk intermediate manufacturing solution to Drug Product solution consisting of 10 mg/mLh teclistamab, 15 miM Acetate, 8% (w/v) Sucrose, 20 μg/mL, EDTA, pH 5.2

Dilution Buffer

Provided herein is a tabular summary of the composition of the Dilution Buffer in Table 16. Final filtration of the dilution buffer was achieved using a sterile 0.22 μm filter.

TABLE 16
Composition of Dilution Buffer

| Component | Composition | | Amount per mL | |
|---|---|---|---|---|
| Sodium acetate trihydrate | 15 | mM | 1.497 | mg |
| Glacial acetic acid | | | 0.240 | mg |
| Sucrose | 8% (w/v) | | 8 | mg |
| Polysorbate 20 | 0.04% | | 0.40 | mg |
| EDTA Disodium salt, Dihydrate | 20 | μg/mL | 0.02 | mg |

| | | |
|---|---|---|
| Water for Injection | q.s to 1.0 mL | q.s to 1.0 mL |

### Preparation of Teclistamab 10 mg/mL Drug Product Solution

Dilution Buffer was added to the 90 mg/mL FB yielding a 10 mg/mL Drug Product solution consisting of 10 mg/mL teclistamab in 15 mM Acetate, 8% (w/v) Sucrose, 0.04% Polysorbate 20, 20 μg/mL EDTA, pH 5.2.

### Example 9: 10 mg/mL Drug Product Formulation: Composition and Components of Primary Packaging

Provided herein is a tabular summary of the composition of the 10 mg/mL Teclistamab Drug Product Formulation in Table 17.

TABLE 17
Composition of 10 mg/mL Teclistamab Drug Product

| Component | Amount per mL | |
|---|---|---|
| Teclistamab | 10 | mg |
| Sodium acetate trihydrate | 1.497 | mg |
| Glacial acetic acid | 0.240 | mg |
| Sucrose | 8 | mg |
| Polysorbate 20 | 0.40 | mg |
| EDTA Disodium salt, Dihydrate | 0.02 | mg |

| | |
|---|---|
| Water for Injection | q.s to 1.0 mL |

The 10 mg/mL teclistamab drug product (DP) primary packaging consists of a glass vial, a polymer vial stopper, and an aluminum seal. Table 18 lists specific components for the primary packaging material of the 30 mg DP presentation.

TABLE 18
10 mg/mL DP Primary packaging material components

| Component | Description |
|---|---|
| Glass vial | 6R glass Type 1 borosilicate |
| Stopper | 20 mm Stopper 4023/50 Gray with Flurotec Coating, RTS |
| Seals | 20 mm flip-off ROYBLU 1280 Flush Button |

### Example 10: Description of 90 mg/mL Stability Study

This study was conducted to monitor teclistamab 90 mg/mL Drug Product attributes placed on stability under various environmental conditions and lengths of time. Study test articles were prepared by aliquoting Formulated Bulk into 2 mL vials at a fill volume of 2.0 mL. The vials were stoppered, capped, and crimp sealed

All studies were to be performed with vials in an inverted orientation.

TABLE 19
Study parameters

| Stability Classification | Storage condition | Duration (Months) |
|---|---|---|
| Real-time | 5 ± 3° C. | 36 |
| Accelerated | 25 ± 2° C./60% RH | 12 |
| Stressed | 40 ± 2° C./75% RH | 6 |

Stability Study Results

The stability results for teclistamab DP held under recommended, accelerated, and stressed conditions are listed below. At all-time points for DP held at recommended storage conditions, all test parameter result values observed per assay study exceeded the criteria consistent with the most preferred embodiment of the stability when held after storage for about 12 months or more and at a temperature of about 5° C., and/or after storage for about 2 years or more and at a temperature of about 5° C. Similarly, peptide map results showed little to no consequential change over time in the measured percent of post translational modification.

Results for teclistamab DP held at accelerated (12 months at 25±2° C./60% RH) and stressed (6 months at 40±2° C./75% RH) conditions showed the expected rates of degradation for Drug Product exposed to prolonged accelerated and stressed storage conditions. Of particular note, the majority of test parameter result values observed per assay study for DP held at accelerated conditions (25° C.) for 12 months showed results consistent with or exceeded the most preferred embodiment of the stability with all but one of the remaining results consistent with the preferred or described embodiments.

TABLE 20
Stability Results for 90 mg/mL Teclistamab Drug Product Stored at 5° C.

| | | | | Particulate Matter | | | |
|---|---|---|---|---|---|---|---|
| | | | | Sub-visible | | | |
| | | | | ≥10 μm: | ≥25 μm: | cSDS (Reduced) | |
| | | | Turbidity | particles | particles | Purity: | new |
| Months | Color of Solution | pH | (NTU) | per vial | per vial | % | peaks |
| 0 | ≤BY6, ≤B6, ≤Y6 | 5.18 | 4.7 | 46 | 1.74 | 98.97 | No new peak > 1.0% |
| | | | | | | | compared to |
| | | | | | | | Reference Material |
| 3 | ≤BY6, ≤B6, ≤Y6 | 5.27 | 5.1 | 40.40 | 0.94 | 99.002 | No new peak > 1.0% |
| | | | | | | | compared to |
| | | | | | | | Reference Material |
| 6 | ≤BY6, ≤B6, ≤Y6 | 5.26 | 5.0 | 67.20 | 1.86 | 98.902 | No new peak > 1.0% |
| | | | | | | | compared to |
| | | | | | | | Reference Material |
| 9 | ≤BY6, ≤B6, ≤Y5 | 5.25 | 5.1 | 36.80 | 3.34 | 98.601 | No new peak > 1.0% |
| | | | | | | | compared to |
| | | | | | | | Reference Material |
| 12 | ≤B6, ≤BY6, ≤Y6 | 5.23 | 5.1 | 86.00 | 6.00 | 98.948 | No new peak > 1.0% |
| | | | | | | | compared to |
| | | | | | | | Reference Material |
| 18 | ≤B6, ≤BY5, ≤Y5 | 5.24 | 5.7 | 44.14 | 2.00 | 98.859 | No new peak > 1.0% |
| | | | | | | | compared to |
| | | | | | | | Reference Material |
| 24 | ≤BY6, ≤B6, ≤Y5 | 5.3 | 5.6 | 21 | 1 | 98.9 | No new peak > 1.0% |
| | | | | | | | compared to |
| | | | | | | | Reference Material |
| 36 | | | | | | | |

| | cSDS (Non-reduced) | | SE HPLC | | | Protein Conc. | Potency T-Cell |
|---|---|---|---|---|---|---|---|
| | Purity | New | Main | HMWS | LMWS | by A₂₈₀ | Activation Assay |
| Months | (%) | Peaks (%) | Component (%) | (%) | (%) | (mg/mL) | % of RM activity |
| 0 | 98.5749 | No new peak > 1.0% | 98.623 | 1.377 | <0.1 | 90.675 | 102 |
| | | compared to | | | | | |
| | | Reference Material | | | | | |
| 3 | 97.481 | No new peak > 1.0% | 98.581 | 1.419 | <0.1 | 90.970 | 96 |
| | | compared to | | | | | |
| | | Reference Material | | | | | |
| 6 | 98.510 | No new peak > 1.0% | 98.534 | 1.466 | <0.1 | 90.295 | 98 |
| | | compared to | | | | | |
| | | Reference Material | | | | | |
| 9 | 98.476 | No new peak > 1.0% | 98.471 | 1.529 | <0.1 | 90.591 | 93 |
| | | compared to | | | | | |
| | | Reference Material | | | | | |
| 12 | 98.674 | No new peak > 1.0% | 98.422 | 1.578 | <0.1 | 90.422 | 82 |
| | | compared to | | | | | |
| | | Reference Material | | | | | |
| 18 | 98.537 | No new peak > 1.0% | 98.283 | 1.717 | <0.1 | 90.717 | 78 |
| | | compared to | | | | | |
| | | Reference Material | | | | | |
| 24 | 98.1 | No new peak > 1.0% | 98.2 | 1.8 | <0.1 | 90.9 | 86 |
| | | compared to | | | | | |
| | | Reference Material | | | | | |
| 36 | | | | | | | |

| | IE-HPLC | | | | | |
|---|---|---|---|---|---|---|
| | Main | Sum of | Sum of | Residual Parental | Residual Parental | |
| | peak | acidic | Basic | Homodimers | Homodimers | Polysorbate |
| Months | (%) | peaks (%) | peaks (%) | Component 1 (%) | Component 2 (%) | 20 (%) |
| 0 | 78.614 | 18.151 | 3.235 | 0.0 | 1.7762 | 0.037 |
| 3 | 78.355 | 18.257 | 3.388 | 0.0000 | 1.6208 | 0.035 |
| 6 | 77.203 | 19.428 | 3.369 | 0.0000 | 1.6008 | 0.038 |
| 9 | 77.240 | 19.064 | 3.695 | 0.0000 | 1.8243 | 0.035 |
| 12 | 75.984 | 20.070 | 3.946 | 0.0000 | 1.3635 | 0.032 |
| 18 | 76.408 | 19.882 | 3.710 | 0.0000 | 1.4543 | 0.032 |
| 24 | 76.7 | 19.7 | 3.7 | 0.0000 | 1.1024 | 0.0304 |
| 36 | | | | | | |

| | Post Translational Modification | | |
|---|---|---|---|
| | BCMA HC Met253/CD3 | CD3 HC Asn103/Asn106 | BCMA HC Asp 101 |
| | HC Met257 Oxidation | Deamidation | Isomerization |
| Months | (%) | (%) | (%) |
| 0 | 3.4 | 12.6 | 5.4 |
| 3 | 3.1 | 12.2 | 6.9 |
| 6 | 3.5 | 11.1 | 5.8 |
| 9 | N/A* | N/A* | N/A* |
| 12 | 3.3 | 12.8 | 8.1 |
| 18 | 3.2 | 12.5 | 8.7 |
| 24 | 3.0 | 11.9 | 8.2 |
| 36 | | | |

*scheduled testing for time point cancelled.

TABLE 21
Stability Results for 90 mg/mL Teclistamab Drug Product Stored at 25° C.

| | | | | Particulate Matter | | | |
|---|---|---|---|---|---|---|---|
| | | | | Sub-visible | | | |
| | | | | ≥10 μm: | ≥25 μm: | cSDS (Reduced) | |
| | | | Turbidity | particles | particles | Purity: | new |
| Months | Color of Solution | pH | (NTU) | per vial | per vial | % | peaks |
| 0 | ≤BY6, ≤B6, ≤Y6 | 5.18 | 4.7 | 46 | 1.74 | 98.97 | No new peak > 1.0% |
| | | | | | | | compared to |
| | | | | | | | Reference Material |
| 1 | ≤BY6, ≤B6, ≤Y5 | 5.27 | 5.2 | 11.46 | 0.00 | 98.832 | No new peak > 1.0% |
| | | | | | | | compared to |
| | | | | | | | Reference Material |
| 3 | ≤BY6, ≤B6, ≤Y6 | 5.29 | 7.6 | 63.46 | 1.46 | 98.397 | No new peak > 1.0% |
| | | | | | | | compared to |
| | | | | | | | Reference Material |
| 6 | ≤BY6, ≤B6, ≤Y5 | 5.30 | 5.2 | 24.54 | 0.66 | 97.679 | No new peak > 1.0% |
| | | | | | | | compared to |
| | | | | | | | Reference Material |
| 9 | ≤BY6, ≤B6, ≤Y6 | 5.27 | 5.0 | 42.66 | 0.66 | 97.458 | No new peak > 1.0% |
| | | | | | | | compared to |
| | | | | | | | Reference Material |
| 12 | ≤BY5, ≤B6, ≤Y6 | 5.2 | 5.8 | 56 | 1 | 96.6 | No new peak > 1.0% |
| | | | | | | | compared to |
| | | | | | | | Reference Material |

| | cSDS (Non-reduced) | | SE HPLC | | | Protein Conc. | Potency T-Cell |
|---|---|---|---|---|---|---|---|
| | Purity | New | Main | HMWS | LMWS | by A₂₈₀ | Activation Assay |
| Months | (%) | Peaks (%) | Component (%) | (%) | (%) | (mg/mL) | % of RM activity |
| 0 | 98.5749 | No new peak > 1.0% | 98.623 | 1.377 | <0.1 | 90.675 | 102 |
| | | compared to | | | | | |
| | | Reference Material | | | | | |
| 1 | 97.333 | No new peak > 1.0% | 98.336 | 1.664 | <0.1 | 90.211 | 94 |
| | | compared to | | | | | |
| | | Reference Material | | | | | |
| 3 | 98.016 | No new peak > 1.0% | 98.086 | 1.914 | <0.1 | 89.789 | 86 |
| | | compared to | | | | | |
| | | Reference Material | | | | | |
| 6 | 97.532 | No new peak > 1.0% | 97.706 | 2.239 | 0.054 | 90.253 | 53 |
| | | compared to | | | | | |
| | | Reference Material | | | | | |
| 9 | 95.310 | No new peak > 1.0% | 97.443 | 2.480 | 0.077 | 90.759 | |
| | | compared to | | | | | |
| | | Reference Material | | | | | |
| 12 | 95.6 | No new peak > 1.0% | 97 | 2.9 | 0.1 | 91.1 | 36 |
| | | compared to | | | | | |
| | | Reference Material | | | | | |

| | IE-HPLC | | | | | |
|---|---|---|---|---|---|---|
| | Main | Sum of | Sum of | Residual Parental | Residual Parental | |
| | peak | acidic | Basic | Homodimers | Homodimers | Polysorbate |
| Months | (%) | peaks (%) | peaks (%) | Component 1 (%) | Component 2 (%) | 20 (%) |
| 0 | 78.614 | 18.151 | 3.235 | 0.0 | 1.7762 | 0.037 |
| 1 | 76.315 | 19.929 | 3.756 | 0.0000 | 1.6930 | 0.034 |
| 3 | 74.789 | 21.034 | 4.177 | 0.0000 | 1.9191 | 0.031 |
| 6 | 70.185 | 24.687 | 5.129 | 0.0000 | 2.2926 | 0.033 |
| 9 | 68.524 | 25.735 | 5.741 | 0.0000 | 2.6209 | 0.034 |
| 12 | 63.3 | 30.4 | 6.3 | 0.0000 | 2.99994 | 0.0358 |

| | Post Translational Modification | | |
|---|---|---|---|
| | BCMA HC Met253/CD3 | CD3 HC Asn103/Asn106 | BCMA HC Asp 101 |
| | HC Met257 Oxidation | Deamidation | Isomerization |
| Months | (%) | (%) | (%) |
| 0 | 3.4 | 12.6 | 5.4 |
| 3 | 3.9 | 14.6 | 17.0 |
| 6 | 4.6 | 14.0 | 22.9 |
| 9 | N/A* | N/A* | N/A* |
| 12 | 4.8 | 19.4 | 24.2 |

*scheduled testing for time point cancelled.

TABLE 22
Stability Results for 90 mg/mL Teclistamab Drug Product Stored at 40° C.

| | | | | Particulate Matter | | | |
|---|---|---|---|---|---|---|---|
| | | | | Sub-visible | | | |
| | | | | ≥10 μm: | ≥25 μm: | cSDS (Reduced) | |
| | | | Turbidity | particles | particles | Purity: | new |
| Months | Color of Solution | pH | (NTU) | per vial | per vial | % | peaks |
| 0 | ≤BY6, ≤B6, ≤Y6 | 5.18 | 4.7 | 46 | 1.74 | 98.97 | No new peak > 1.0% |
| | | | | | | | compared to |
| | | | | | | | Reference Material |
| 1 | ≤B6, ≤BY5, ≤Y5 | 5.30 | 5.6 | 51.74 | 1.74 | 97.856 | No new peak > 1.0% |
| | | | | | | | compared to |
| | | | | | | | Reference Material |
| 3 | ≤B6, ≤BY6, ≤Y5 | 5.27 | 7.1 | 134.14 | 7.06 | 94.740 | No new peak > 1.0% |
| | | | | | | | compared to |
| | | | | | | | Reference Material |
| 6 | ≤B5, ≤BY5, ≤Y5 | 5.34 | 9.7 | 279.20 | 14.00 | 90.948 | No new peak > 1.0% |
| | | | | | | | compared to |
| | | | | | | | Reference Material |

| | cSDS (Non-reduced) | | SE HPLC | | | Protein Conc. | Potency T-Cell |
|---|---|---|---|---|---|---|---|
| | Purity | New | Main | HMWS | LMWS | by A₂₈₀ | Activation Assay |
| Months | (%) | Peaks (%) | Component (%) | (%) | (%) | (mg/mL) | % of RM activity |
| 0 | 98.5749 | No new peak > 1.0% | 98.623 | 1.377 | <0.1 | 90.675 | 102 |
| | | compared to | | | | | |
| | | Reference Material | | | | | |
| 1 | 95.340 | No new peak > 1.0% | 97.105 | 2.787 | <0.1 | 90.042 | 53 |
| | | compared to | | | | | |
| | | Reference Material | | | | | |
| 3 | 92.741 | No new peak > 1.0% | 93.835 | 5.864 | <0.1 | 90.549 | 20 |
| | | compared to | | | | | |
| | | Reference Material | | | | | |
| 6 | 86.984 | No new peak > 1.0% | 85.581 | 13.781 | <0.1 | 90.127 | 21 |
| | | compared to | | | | | |
| | | Reference Material | | | | | |

| | IE-HPLC | | | | | |
|---|---|---|---|---|---|---|
| | Main | Sum of | Sum of | Residual Parental | Residual Parental | |
| | peak | acidic | Basic | Homodimers | Homodimers | Polysorbate |
| Months | (%) | peaks (%) | peaks (%) | Component 1 (%) | Component 2 (%) | 20 (%) |
| 0 | 78.614 | 18.151 | 3.235 | 0.0 | 1.7762 | 0.037 |
| 1 | 69.409 | 24.879 | 5.712 | 0.0000 | 2.5190 | 0.031 |
| 3 | 54.371 | 35.690 | 9.939 | 0.0000 | 4.3034 | 0.026 |
| 6 | 34.730 | 47.853 | 17.417 | 0.0000 | 9.8809 | 0.024 |

| | Post Translational Modification | | |
|---|---|---|---|
| | BCMA HC Met253/CD3 | CD3 HC Asn103/Asn106 | BCMA HC Asp 101 |
| | HC Met257 Oxidation | Deamidation | Isomerization |
| Months | (%) | (%) | (%) |
| 0 | 3.4 | 12.6 | 5.4 |
| 1 | 4.2 | 17.2 | 24.3 |
| 3 | 6.6 | 25.5 | 48.6 |
| 6 | 13.2 | 36.6 | 60.8 |

### Example 11: Description of 10 mg/mL Stability Study

This study was conducted to monitor teclistamab 10 mg/mLh Drug Product attributes placed on stability under various environmental conditions and lengths of time. Study test articles were prepared by aliquoting Formulated Bulk into 6R vials at a fill volume of 3.5 mL.

The vials were stoppered, capped, and crimp sealed

All studies were to be performed with vials in an inverted orientation.

TABLE 23
Study parameters

| Stability Classification | Storage condition | Duration (Months) |
|---|---|---|
| Real-time | 5 ± 3° C. | 36 |
| Accelerated | 25 ± 2° C./60% RH | 12 |
| Stressed | 40 ± 2° C./75% RH | 6 |

Stability Study Results

The stability results for teclistamab DP held under recommended, accelerated, and stressed conditions are listed below. At all-time points for DP held at recommended storage conditions, all test parameter result values observed per assay study exceeded the criteria consistent with the most preferred embodiment of the stability when held after storage for about 12 months or more and at a temperature of about 5° C., and/or after storage for about 2 years or more and at a temperature of about 5° C. Similarly, peptide map results showed little to no consequential change over time in the measured percent of post translational modification.

Results for teclistamab DP held at accelerated (25±2° C./60% RH) and stressed (40±2° C./75% RH) conditions showed the expected rates of degradation for Drug Product exposed to prolonged accelerated and stressed storage conditions. Of particular note, the majority of test parameter result values observed per assay study for DP held at accelerated conditions (25° C.) for 12 months showed results consistent with or exceeded the most preferred embodiment of the stability with all but one of the remaining results consistent with the preferred or described embodiments

TABLE 24
Stability Results for 10 mg/mL Teclistamab Drug Product Stored at 5° C.

| | | | | Particulate Matter | | | |
|---|---|---|---|---|---|---|---|
| | | | | Sub-visible | | | |
| | | | | ≥10 μm: | ≥25 μm: | cSDS (Reduced) | |
| | | | Turbidity | particles | particles | Purity: | new |
| Months | Color of Solution | pH | (NTU) | per vial | per vial | % | peaks |
| 0 | ≤B9, ≤BY7, ≤Y7 | 5.18 | 4.1 | 38.25 | 0.70 | 98.843 | No new peak > 1.0% |
| | | | | | | | compared to |
| | | | | | | | Reference Material |
| 3 | ≤B9, ≤BY7, ≤Y7 | 5.11 | 4.3 | 66.04 | 3.50 | 98.885 | No new peak > 1.0% |
| | | | | | | | compared to |
| | | | | | | | Reference Material |
| 6 | ≤B8, ≤BY7, ≤Y7 | 5.12 | 4.0 | 25.65 | 0.45 | 98.858 | No new peak > 1.0% |
| | | | | | | | compared to |
| | | | | | | | Reference Material |
| 9 | ≤B8, ≤BY7, ≤Y6 | 5.19 | 4.1 | 30.80 | 0.70 | 98.919 | No new peak > 1.0% |
| | | | | | | | compared to |
| | | | | | | | Reference Material |
| 12 | ≤B9, ≤BY7, ≤Y7 | 5.17 | 4.2 | 31.04 | 0.94 | 98.810 | No new peak > 1.0% |
| | | | | | | | compared to |
| | | | | | | | Reference Material |
| 18 | ≤B8, ≤BY7, ≤Y7 | 5.17 | 4.8 | 37.55 | 0.70 | 98.828 | No new peak > 1.0% |
| | | | | | | | compared to |
| | | | | | | | Reference Material |
| 24 | ≤B6, ≤BY6, ≤Y6 | 5.2 | 4.1 | 38 | 0 | 98.7 | No new peak > 1.0% |
| | | | | | | | compared to |
| | | | | | | | Reference Material |
| 36 | | | | | | | |

| | cSDS (Non-reduced) | | SE HPLC | | | Protein Conc. | Potency T-Cell |
|---|---|---|---|---|---|---|---|
| | Purity | New | Main | HMWS | LMWS | by A₂₈₀ | Activation Assay |
| Months | (%) | Peaks (%) | Component (%) | (%) | (%) | (mg/mL) | % of RM activity |
| 0 | 98.573 | No new peak > 1.0% | 99.352 | 0.648 | <0.1 | 9.763 | 95 |
| | | compared to | | | | | |
| | | Reference Material | | | | | |
| 3 | 98.635 | No new peak > 1.0% | 99.359 | 0.641 | <0.1 | 9.800 | 104 |
| | | compared to | | | | | |
| | | Reference Material | | | | | |
| 6 | 98.574 | No new peak > 1.0% | 99.274 | 0.726 | <0.1 | 9.684 | 93 |
| | | compared to | | | | | |
| | | Reference Material | | | | | |
| 9 | 97.933 | No new peak > 1.0% | 99.293 | 0.707 | <0.1 | 9.926 | 91 |
| | | compared to | | | | | |
| | | Reference Material | | | | | |
| 12 | 98.139 | No new peak > 1.0% | 99.299 | 0.701 | <0.1 | 10.011 | 98 |
| | | compared to | | | | | |
| | | Reference Material | | | | | |
| 18 | 98.589 | No new peak > 1.0% | 99.257 | 0.743 | <0.1 | 9.831 | 90 |
| | | compared to | | | | | |
| | | Reference Material | | | | | |
| 24 | 98.5 | No new peak > 1.0% | 99.2 | 0.8 | <0.1 | 9.7 | 87 |
| | | compared to | | | | | |
| | | Reference Material | | | | | |
| 36 | | | | | | | |

| | IE-HPLC | | | | | |
|---|---|---|---|---|---|---|
| | Main | Sum of | Sum of | Residual Parental | Residual Parental | |
| | peak | acidic | Basic | Homodimers | Homodimers | Polysorbate |
| Months | (%) | peaks (%) | peaks (%) | Component 1 (%) | Component 2 (%) | 20 (%) |
| 0 | 76.645 | 19.958 | 3.397 | 0.0000 | 1.6503 | 0.038 |
| 3 | 76.722 | 19.975 | 3.303 | 0.0000 | 1.6604 | 0.039 |
| 6 | 76.292 | 20.303 | 3.405 | 0.0000 | 1.3380 | 0.038 |
| 9 | 75.040 | 21.318 | 3.642 | 0.0000 | 1.0150 | 0.038 |
| 12 | 75.623 | 20.627 | 3.750 | 0.0000 | 1.4741 | 0.038 |
| 18 | 74.580 | 21.464 | 3.956 | 0.0000 | 1.1939 | 0.038 |
| 24 | 75.1 | 21.3 | 3.6 | 0.0000 | 0.9577 | 0.0378 |
| 36 | | | | | | |

| | Post Translational Modification | | |
|---|---|---|---|
| | BCMA HC Met253/CD3 | CD3 HC Asn103/Asn106 | BCMA HC Asp 101 |
| | HC Met257 Oxidation | Deamidation | Isomerization |
| Months | (%) | (%) | (%) |
| 0 | 3.9 | 13.3 | 5.3 |
| 3 | 3.1 | 13.4 | 5.6 |
| 6 | 3.8 | 13.4 | 7.2 |
| 9 | 4.8 | 14.3 | 7.0 |
| 12 | 3.6 | 13.1 | 8 |
| 18 | 3.3 | 13.0 | 8.7 |
| 24 | 3.7 | 13.1 | 9.1 |
| 36 | | | |

TABLE 25
Stability Results for 10 mg/mL Teclistamab Drug Product Stored at 25° C.

| | | | | Particulate Matter | | | |
|---|---|---|---|---|---|---|---|
| | | | | Sub-visible | | | |
| | | | | ≥10 μm: | ≥25 μm: | cSDS (Reduced) | |
| | | | Turbidity | particles | particles | Purity: | new |
| Months | Color of Solution | pH | (NTU) | per vial | per vial | % | peaks |
| 0 | ≤B9, ≤BY7, ≤Y7 | 5.18 | 4.1 | 38.25 | 0.70 | 98.843 | No new peak > 1.0% |
| | | | | | | | compared to |
| | | | | | | | Reference Material |
| 1 | ≤B9, ≤BY7, ≤Y7 | 5.12 | 3.9 | 22.64 | 0.00 | 98.912 | No new peak > 1.0% |
| | | | | | | | compared to |
| | | | | | | | Reference Material |
| 3 | ≤B9, ≤BY7, ≤Y7 | 5.12 | 4.0 | 47.35 | 0.70 | 98.348 | No new peak > 1.0% |
| | | | | | | | compared to |
| | | | | | | | Reference Material |
| 6 | ≤B9, ≤BY7, ≤Y7 | 5.21 | 4.2 | 42.70 | 0.24 | 97.884 | No new peak > 1.0% |
| | | | | | | | compared to |
| | | | | | | | Reference Material |
| 9 | ≤B9, ≤BY7, ≤Y7 | 5.22 | 4.3 | 41.75 | 0.24 | 97.392 | No new peak > 1.0% |
| | | | | | | | compared to |
| | | | | | | | Reference Material |
| 12 | ≤BY6, ≤B7, ≤Y6 | 5.2 | 3.8 | 6.8 | 0.0 | 96.7 | No new peak > 1.0% |
| | | | | | | | compared to |
| | | | | | | | Reference Material |

| | cSDS (Non-reduced) | | SE HPLC | | | Protein Conc. | Potency T-Cell |
|---|---|---|---|---|---|---|---|
| | Purity | New | Main | HMWS | LMWS | by A₂₈₀ | Activation Assay |
| Months | (%) | Peaks (%) | Component (%) | (%) | (%) | (mg/mL) | % of RM activity |
| 0 | 98.573 | No new peak > 1.0% | 99.352 | 0.648 | <0.1 | 9.763 | 95 |
| | | compared to | | | | | |
| | | Reference Material | | | | | |
| 1 | 97.625 | No new peak > 1.0% | 99.239 | 0.761 | <0.1 | 9.715 | 94 |
| | | compared to | | | | | |
| | | Reference Material | | | | | |
| 3 | 97.820 | No new peak > 1.0% | 99.075 | 0.925 | <0.1 | 9.694 | 88 |
| | | compared to | | | | | |
| | | Reference Material | | | | | |
| 6 | 96.069 | No new peak > 1.0% | 98.988 | 0.950 | 0.062 | 9.831 | 59 |
| | | compared to | | | | | |
| | | Reference Material | | | | | |
| 9 | 96.489 | No new peak > 1.0% | 98.872 | 1.045 | 0.083 | 9.974 | 45 |
| | | compared to | | | | | |
| | | Reference Material | | | | | |
| 12 | 96.4 | No new peak > 1.0% | 98.7 | 1.2 | 0.1 | 9.8 | 36 |
| | | compared to | | | | | |
| | | Reference Material | | | | | |

| | IE-HPLC | | | | | |
|---|---|---|---|---|---|---|
| | Main | Sum of | Sum of | Residual Parental | Residual Parental | |
| | peak | acidic | Basic | Homodimers | Homodimers | Polysorbate |
| Months | (%) | peaks (%) | peaks (%) | Component 1 (%) | Component 2 (%) | 20 (%) |
| 0 | 76.645 | 19.958 | 3.397 | 0.0000 | 1.6503 | 0.038 |
| 1 | 76.412 | 20.152 | 3.436 | 0.0000 | 1.3668 | 0.038 |
| 3 | 73.018 | 23.108 | 3.874 | 0.0000 | 1.2430 | 0.038 |
| 6 | 67.970 | 27.495 | 4.535 | 0.0000 | 1.1820 | 0.037 |
| 9 | 65.021 | 29.970 | 5.009 | 0.0000 | 1.8747 | 0.036 |
| 12 | 59.3 | 35.4 | 5.3 | 0.0 | 2.4148 | 0.035 |

| | Post Translational Modification | | |
|---|---|---|---|
| | BCMA HC Met253/CD3 | CD3 HC Asn103/Asn106 | BCMA HC Asp 101 |
| | HC Met257 Oxidation | Deamidation | Isomerization |
| Months | (%) | (%) | (%) |
| 0 | 3.9 | 13.3 | 5.3 |
| 3 | 4.1 | 16.5 | 17.4 |
| 6 | 5.2 | 16.3 | 25.6 |
| 9 | N/A* | N/A* | N/A* |
| 12 | 5.1 | 23.3 | 38.2 |

*scheduled testing for time point cancelled

TABLE 26
Stability Results for 10 mg/mL Teclistamab Drug Product Stored at 40° C.

| | | | | Particulate Matter | | | |
|---|---|---|---|---|---|---|---|
| | | | | Sub-visible | | | |
| | | | | ≥10 μm: | ≥25 μm: | cSDS (Reduced) | |
| | | | Turbidity | particles | particles | Purity: | new |
| Months | Color of Solution | pH | (NTU) | per vial | per vial | % | peaks |
| 0 | ≤B9, ≤BY7, ≤Y7 | 5.18 | 4.1 | 0.038 | 38.25 | 0.70 | No new peak > 1.0% |
| | | | | | | | compared to |
| | | | | | | | Reference Material |
| 1 | ≤B9, ≤BY7, ≤Y7 | 5.14 | 3.6 | 0.037 | 30.55 | 0.45 | No new peak > 1.0% |
| | | | | | | | compared to |
| | | | | | | | Reference Material |
| 3 | ≤B8, ≤BY7, ≤Y7 | 5.16 | 4.3 | 0.034 | 79.55 | 0.45 | No new peak > 1.0% |
| | | | | | | | compared to |
| | | | | | | | Reference Material |
| 6 | ≤B8, ≤BY7, ≤Y6 | 5.27 | 5.3 | 0.033 | 52.04 | 0.24 | No new peak > 1.0% |
| | | | | | | | compared to |
| | | | | | | | Reference Material |

| | cSDS (Non-reduced) | | SE HPLC | | | Protein Conc. | Potency T-Cell |
|---|---|---|---|---|---|---|---|
| | Purity | New | Main | HMWS | LMWS | by A₂₈₀ | Activation Assay |
| Months | (%) | Peaks (%) | Component (%) | (%) | (%) | (mg/mL) | % of RM activity |
| 0 | 98.573 | No new peak > 1.0% | 99.352 | 0.648 | 0.000 | 9.763 | 95 |
| | | compared to | | | | | |
| | | Reference Material | | | | | |
| 1 | 97.580 | No new peak > 1.0% | 98.727 | 1.169 | 0.104 | 9.736 | 58 |
| | | compared to | | | | | |
| | | Reference Material | | | | | |
| 3 | 93.607 | No new peak > 1.0% | 96.316 | 3.338 | 0.345 | 9.678 | 20 |
| | | compared to | | | | | |
| | | Reference Material | | | | | |
| 6 | 90.205 | No new peak > 1.0% | 90.767 | 8.619 | 0.614 | 10.000 | 3 |
| | | compared to | | | | | |
| | | Reference Material | | | | | |

| | IE-HPLC | | | | | |
|---|---|---|---|---|---|---|
| | Main | Sum of | Sum of | Residual Parental | Residual Parental | |
| | peak | acidic | Basic | Homodimers | Homodimers | Polysorbate |
| Months | (%) | peaks (%) | peaks (%) | Component 1 (%) | Component 2 (%) | 20 (%) |
| 0 | 78.614 | 18.151 | 3.235 | 0.0 | 1.7762 | 0.037 |
| 1 | 76.645 | 19.958 | 3.397 | 0.0000 | 1.6503 | 0.038 |
| 3 | 66.527 | 27.923 | 5.550 | 0.0000 | 1.9136 | 0.037 |
| 6 | 44.787 | 47.144 | 8.069 | 0.3452 | 2.6502 | 0.034 |

| | Post Translational Modification | | |
|---|---|---|---|
| | BCMA HC Met253/CD3 | CD3 HC Asn103/Asn106 | BCMA HC Asp 101 |
| | HC Met257 Oxidation | Deamidation | Isomerization |
| Months | (%) | (%) | (%) |
| 0 | 3.9 | 13.3 | 5.3 |
| 1 | 4.1 | 19.7 | 23.3 |
| 3 | 7.4 | 35.2 | 50.9 |
| 6 | 14.5 | 47.1 | 62.0 |

1. A stable aqueous pharmaceutical composition comprising:
a) a concentration of about 7.5 mg/mL to about 12.5 mg/mL of a bispecific B-cell mature antigen (BCMA)/cluster of differentiation 3 (CD3) antibody or antigen-binding fragment thereof, the bispecific BCMA/CD3 antibody or antigen-binding fragment thereof comprising:
(1) a first heavy chain (HCl) comprising a HCl variable region 1 (VH1), wherein the VH1 comprises a heavy chain complementarity determining region 1 (HCDR1), a HCDR2, and a HCDR3 having the amino acid sequences of SEQ ID NOs:1, 2, and 3, respectively;
(2) a first light chain (LC1) comprising a LC1 variable region (VL1), wherein the VL1 comprises a light chain complementarity determining region 1 (LCDR1), a LCDR2, and a LCDR3 having the amino acid sequences of SEQ ID NOs: 4, 5, and 6, respectively;
(3) a second heavy chain (HC2) comprising a HC2 variable region 2 (VH2), wherein the VH2 comprises a heavy chain complementarity determining region 1 (HCDR1), a HCDR2, and a HCDR3 having the amino acid sequences of SEQ ID NOs:11, 12, and 13, respectively; and
(4) a second light chain (LC2) comprising a LC2 variable region 2 (VL2), wherein the VL2 comprises a light chain complementarity determining region 1 (LCDR1), a LCDR2, and a LCDR3 having the amino acid sequences of SEQ ID NOs:14, 15, and 16, respectively;
b) about 10 mM to about 20 mM of acetate and/or pharmaceutically acceptable acetate salt;
c) about 6% (w/v) to about 10% (w/v) of sucrose;
d) about 16 μg/mL to about 24 μg/mL of ethylenediaminetetraacetic acid (EDTA);
e) about 0.01% to about 0.07% polysorbate 20; and
f) a pH from about 4.7 to about 5.7.

2. A stable aqueous pharmaceutical composition comprising:
a) a concentration of about 76.5 mg/mL to about 103.5 mg/mL of a bispecific B-cell mature antigen (BCMA)/cluster of differentiation 3 (CD3) antibody or antigen-binding fragment thereof, the bispecific BCMA/CD3 antibody or antigen-binding fragment thereof comprising:
(1) a first heavy chain (HCl) comprising a HCl variable region 1 (VH1), wherein the VH1 comprises a heavy chain complementarity determining region 1 (HCDR1), a HCDR2, and a HCDR3 having the amino acid sequences of SEQ ID NOs:1, 2, and 3, respectively;
(2) a first light chain (LC1) comprising a LC1 variable region (VL1), wherein the VL1 comprises a light chain complementarity determining region 1 (LCDR1), a LCDR2, and a LCDR3 having the amino acid sequences of SEQ ID NOs: 4, 5, and 6, respectively;
(3) a second heavy chain (HC2) comprising a HC2 variable region 2 (VH2), wherein the VH2 comprises a heavy chain complementarity determining region 1 (HCDR1), a HCDR2, and a HCDR3 having the amino acid sequences of SEQ ID NOs:11, 12, and 13, respectively; and
(4) a second light chain (LC2) comprising a LC2 variable region 2 (VL2), wherein the VL2 comprises a light chain complementarity determining region 1 (LCDR1), a LCDR2, and a LCDR3 having the amino acid sequences of SEQ ID NOs:14, 15, and 16, respectively;
b) about 10 mM to about 20 mM of acetate and/or pharmaceutically acceptable acetate salt;
c) about 6% (w/v) to about 10% (w/v) of sucrose;
d) about 16 μg/mL to about 24 μg/mL of ethylenediaminetetraacetic acid (EDTA);
e) about 0.01% to about 0.07% polysorbate 20; and
f) a pH from about 4.7 to about 5.7.

3. The stable aqueous pharmaceutical composition of claim 2, wherein the bispecific BCMA/CD3 antibody comprises a VH1 having the amino acid sequence of SEQ ID NO:7, and a VL1 having the amino acid sequence of SEQ ID NO:8.

4. The stable aqueous pharmaceutical composition of claim 2, wherein the bispecific BCMA/CD3 antibody comprises a HC1 having the amino acid sequence of SEQ ID NO:9, and a LC1 having the amino acid sequence of SEQ ID NO:10.

5. The stable aqueous pharmaceutical composition of claim 2, wherein the bispecific BCMA/CD3 antibody comprises a VH2 having the amino acid sequence of SEQ ID NO:17, and a VL2 having the amino acid sequence of SEQ ID NO:18.

6. The stable aqueous pharmaceutical composition of claim 2, wherein the bispecific BCMA/CD3 antibody comprises a HC2 having the amino acid sequence of SEQ ID NO:19, and a LC2 having the amino acid sequence of SEQ ID NO:20.

7. The stable aqueous pharmaceutical composition of claim 2, wherein the bispecific BCMA/CD3 antibody is teclistamab.

8. The stable aqueous pharmaceutical composition of claim 1, wherein the bispecific BCMA/CD3 antibody has a concentration of about 8 mg/mL to about 12 mg/mL.

9. The stable aqueous pharmaceutical composition of claim 8, wherein the bispecific BCMA/CD3 antibody has a concentration of about 9 mg/mL to about 11 mg/mL.

10. The stable aqueous pharmaceutical composition of claim 8, wherein the bispecific BCMA/CD3 antibody has a concentration of about 10 mg/mL.

11. The stable aqueous pharmaceutical composition of claim 2, wherein the bispecific BCMA/CD3 antibody has a concentration of about 85 mg/mL to about 95 mg/mL.

12. The stable aqueous pharmaceutical composition of claim 11, wherein the bispecific BCMA/CD3 antibody has a concentration of about 87 mg/mL to about 93 mg/mL.

13. The stable aqueous pharmaceutical composition of claim 12, wherein the bispecific BCMA/CD3 antibody has a concentration of about 90 mg/mL.

14. The stable aqueous pharmaceutical composition of claim 2, wherein the composition comprises about 12 mM to about 18 mM of acetate and/or pharmaceutically acceptable acetate salt.

15. (canceled)

16. The stable aqueous pharmaceutical composition of claim 2, wherein the composition comprises about 15 mM of acetate and/or pharmaceutically acceptable acetate salt.

17. The stable aqueous pharmaceutical composition of claim 2, wherein the composition comprises about 7% (w/v) to about 9% (w/v) of sucrose.

18. The stable aqueous pharmaceutical composition of claim 17, wherein the composition comprises about 8% (w/v) of sucrose.

19. The stable aqueous pharmaceutical composition of claim 2, wherein the composition comprises about 18 μg/mL to about 22 μg/mL of EDTA.

20. The stable aqueous pharmaceutical composition of claim 2, wherein the composition comprises about 20 μg/mL of EDTA.

21. The stable aqueous pharmaceutical composition of claim 2, wherein the composition comprises about 0.02% to about 0.06% of polysorbate 20 (PS-20).

22. (canceled)

23. The stable aqueous pharmaceutical composition of claim 21, wherein the composition comprises about 0.04% of PS-20.

24. The stable aqueous pharmaceutical composition of claim 2, wherein the pH is about 4.8 to about 5.6.

25. (canceled)

26. The stable aqueous pharmaceutical composition of claim 24, wherein the pH is about 5.2.

27. The stable aqueous pharmaceutical composition of claim 2, wherein the composition comprises 90 mg/mL of the bispecific BCMA/CD3 antibody, 15 mM of acetate and/or pharmaceutically acceptable acetate salt, 8% (w/v) sucrose, 20 μg/mL of EDTA, 0.04% PS-20, and a pH of 5.2.

28. The stable aqueous pharmaceutical composition of claim 2, wherein the stable aqueous pharmaceutical composition is stable at a temperature of about 2-8° C. for at least two years.

29. The stable aqueous pharmaceutical composition of claim 2, wherein stability isdefined based on color of solution, pH, turbidity, percentage of purity, percentage of new peaks, percentage of main component, percentage of high molecular weight species (HWMS), percentage of of low molecular weight species (LMWS), percentage of sum of acidic peaks, percentage of sum of basic peaks, protein concentration, percentage of T cell activation, percentage of PS-20 (w/v), or any combination thereof.

30. A method of treating cancer in a subject in need thereof, the method comprising administering to the subject the stable aqueous pharmaceutical composition of claim 2.

31. The method of claim 30, wherein the administering is subcutaneous.

32-45. (canceled)
